(19) United States
(12) Patent Application Publication
(10) Pub. No.: US 2012/0027837 A1
(43) Pub. Date: Feb. 2, 2012

(54) MULTILAYER COATING COMPOSITIONS, COATED SUBSTRATES AND METHODS THEREOF

(75) Inventors: Peter C. DeMuth, Cambridge, MA (US); Darrell J. Irvine, Arlington, MA (US); Raymond E. Samuel, Sharon, MA (US); Paula T. Hammond, Newton, MA (US)

(73) Assignee: Massachusetts Institute of Technology, Cambridge, MA (US)

(21) Appl. No.: 13/115,107

(22) Filed: May 24, 2011

Related U.S. Application Data
(60) Provisional application No. 61/368,254, filed on Jul. 27, 2010.
(60) Provisional application No. 61/368,259, filed on Jul. 28, 2010.

(51) Int. Cl. A61K 9/70; B32B 27/08; C12N 11/02; B05D 1/36; B32B 27/34; B32B 27/40; B82Y 30/00

(52) U.S. Cl. 424/443; 428/474.4; 428/475.2; 428/423.5; 435/177; 427/2.14; 977/773

(57) ABSTRACT

The present invention provides, among other things, multilayer film coating compositions, coated substrates and methods thereof In some embodiments, a structure, comprising a substrate and a multilayer film on the substrate, wherein the multilayer film comprises a first plurality of first units, each first unit comprising a protamine polypeptide. In some embodiments, a structure comprising a microneedle substrate and a multilayer film coated on at least portion of the microneedle substrate, wherein the multilayer film comprises an agent for release and a first plurality of first unit; each first unit comprising a first layer and a second layer, wherein the first layer and the second layer are associated with one another.

### CROSS REFERENCE OF RELATED APPLICATIONS

The present application claims priority to U.S. provisional patent applications, U.S. Ser. No. 61/368,254, filed Jul. 27, 2010; and U.S. Ser. No. 61/368,259, filed Jul.28, 2010, the contents of which are incorporated herein by reference.

### GOVERNMENT SUPPORT

This invention was made with government support under Grant No. W911NF-07-D-0004, awarded by the Army Research Office and under Grant No. R01 AG029601, awarded by the National Institutes of Health. The Government of the United States has certain rights in this application.

### BACKGROUND

Layer-by-Layer (LBL) assembly of multilayer film coatings is driven by the alternating deposition of materials (e.g., polymers with complementary electrostatic functionalities). The LbL assembly process produces nanometer to micron scale thin film coatings. A major benefit of LbL assembly is the potential to achieve controlled and sequential delivery of therapeutic agents by tuning the deposition of these agents at specific layers within the film.

There is a particular interest in achieving delivery of vaccines and/or therapeutic agents. Delivery through the skin (i.e., transcutaneous delivery) is a focus of much research. Thus, there is a need in the art for versatile platform for delivery, particularly transcutaneous delivery of drugs and other agents that is effective, generally applicable, safe, pain-free, and/or cost effective.

### SUMMARY

The present invention provides, among other things, certain structures comprising a multilayer film, for example as a coating composition on a substrate.

In one aspect, the invention provides a multilayer film comprising a protamine polypeptide formed by a layer-by-layer technique. Various structures comprising substrates coated with such a multilayer film coating composition are provided.

In one aspect, the invention provides a structure comprising a substrate arranged and constructed for contact with a biological tissue; such a substrate being coated with a multilayer film coating composition. In some embodiments, such a substrate is or comprises a microneedle or a microneedle array.

Among other things, the present invention demonstrates and achieves various improvements in microneedle devices, and particularly in delivery of agents from the devices. The present invention also encompasses the recognition that, in many cases, combining the flexible and highly tunable nature of provided multilayer films with microneedle devices provides a versatile platform for transcutaneous delivery of a variety of agents.

In some embodiments, a multilayer film comprises a first plurality of bilayers. In some embodiments, a multilayer film further comprises a second plurality of bilayers.

In some embodiments, provided multilayer films/structures in the present invention comprise at least an agent to be released. In some embodiments, a composite (e.g., drug-embedded nanoparticles) is incorporated into such a multilayer film/a structure. In some embodiments, a biomolecule (e.g., a plasmid DNA, peptide, etc.) serves as an alternative layer in at least a portion of a multilayer film and can be released. Such a multilayer film and/or a structure comprising the multilayer film on a substrate can be used to deliver/release one or more agents in a sustained and controlled manner.

In some embodiments, provided multilayer films/structures in the present invention comprise a layer of cells (e.g., osteoblastic or pre-osteoblast cells).

Among other things, the present invention provides methods of making and using such a structure and/or a film.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### DEFINITIONS

In order for the present invention to be more readily understood, certain terms are first defined below. Additional definitions for the following terms and other terms are set forth throughout the specification.

“Associated”: As used herein, the terms “associated”, “conjugated”, “linked”, “attached”, “complexed”, and “incorporated,” and grammatic equivalents, typically refer to two or more moieties connected with one another, either directly or indirectly (e.g., via one or more additional moieties that serve as a linking agent), to form a structure that is sufficiently stable so that the moieties remain connected under the conditions in which the structure is used, e.g., physiological conditions. In some embodiments, the moieties are associated to one another by one or more covalent bonds. In some embodiments, the moieties are associated to one another by a mechanism that involves specific (but non-covalent) binding (e.g. streptavidin/avidin interactions, antibody/antigen interactions, etc.). Alternatively or additionally, a sufficient number of weaker interactions (non-covalent) can provide sufficient stability for moieties to remain connected. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible”, as used herein is intended to describe materials that do not elicit a substantial detrimental response in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

“Biodegradable”: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

“Biological tissue”: As used herein, “biological tissue” includes essentially any cells, tissue, or organs, including the skin or parts thereof, mucosal tissues, vascular tissues, lymphatic vessels, ocular tissues (e.g., cornea, conjunctiva, sclera), and cell membranes. The biological tissue can be in humans or other types of animals (particularly mammals), as well as in plants, insects, or other organisms, including bacteria, yeast, fungi, and embryos. Human skin and sclera are biological tissues of particular use with the present microneedle devices and methods of use thereof

“Hydrolytically degradable”: As used herein, “hydrolytically degradable” polymers are polymers that degrade fully in the sole presence of water. In preferred embodiments, the polymers and hydrolytic degradation byproducts are biocompatible. As used herein, the term “non-hydrolytically degradable” refers to polymers that do not fully degrade in the sole presence of water.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polyelectrolyte” or “polyion”: The terms “polyelectrolyte” or “polyion”, as used herein, refer to a polymer which under some set of conditions (e.g., physiological conditions) has a net positive or negative charge. Polycations have a net positive charge and polyanions have a net negative charge. The net charge of a given polyelectrolyte or polyion may depend on the surrounding chemical conditions, e.g., on the pH.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present application, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In some embodiments, a peptide includes one or more residues that contains a pendant moiety such as a glycan (e.g., is a glycopeptide), a PEG moiety (e.g., is a PEGylated polypeptide), etc. In some embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In some embodiments, peptides for use in accordance with the present invention are provided and/or utilized in a form selected from the group consisting of salt forms, crystal forms, and combinations thereof

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Protamine polypeptide”: The term “protamine polypeptide” refers to a polypeptide having an amino acid sequence that defines it as a protamine. Various sequence alignments have been performed of known and/or naturally-occurring protamines, and characteristic sequence elements are established (see, for example, R. Balhorn, “The protamine family of sperm nuclear proteins”, Genome Biology 2007, 8:227, which is incorporated by reference). In some embodiments, one or more of protamine P1 genes is a characteristic sequence element of a protamine. In some embodiments, a protamine polypeptide is a polypeptide whose amino acid sequence includes one or more of the sequences of protamine P2 genes. Alternatively or additionally, in some embodiments, a protamine polypeptide is a polypeptide whose amino acid sequence shows at least about 50%, about 60%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, overall sequence identity with one or more of the reference protamine polypeptide sequences such as protamine P1 genes, protamine P2 gene, etc.; in some such embodiments, the reference protamine polypeptide sequence is a mammalian (e.g., mouse or human) protamine polypeptide.

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Substantial” or “substantive”: As used herein, the terms “substantial” or “substantive” and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Therapeutic agent”, “medication” or “drug”: As used herein, the phrases “therapeutic agent”, “medication”, or “drug” may be used interchangeably. They refer to any agent that, when administered to a subject, has a therapeutic effect and/or elicits a desired biological and/or pharmacological effect.

“Treating:” As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DESCRIPTION OF DRAWING

FIG. 2(A) SEM micrograph of uncoated PLGA microneedle arrays of pyramidal geometry (scale-500 μm). (B) Film growth (left axis) and absorbance (right axis) for Poly-1/pLUCₙ multilayers assembled on silicon/quartz bearin a (PS/SPS)₂₀ initiating layer (black bar-(PS/SPS)₂₀ grey bar-(Poly-1/pLUC)ₙ, dashed line-Ab-260nm. (C, D) Representative confocal micrographs showing a (C) PS/SPS₂₀-(Poly 1/Cy3-pLUC)₂₄ coated microneedle and a (D) (PS/SPS)₂₀-(Poly-1/DiI-PLGA NP)₄ coated microneedle (left-transverse section, right-lateral sections, 200 μm intervals, scale-200 μm). (E) SEM micrograph showing a (PS/SPS)₂₀-(Poly-1/PLGA NP)₄ coated microneedle array (scale-50 μm). (F) Representative confocal micrographs showing a (PS/SPS)₂₀-(Poly-1/Cy3-pLUC)₂₄-(Poly-1/DiD-PLGA NP)₄ co-coated microneedle (transverse and lateral sections, left-3-pLUC, right-DiD-PLGA NP, scale-200 μm)

FIG. 6(A) Optical micrograph of ear skin showing microneedle penetration pattern stained using trypan blue (scale-100 μm). (B) Representative confocal z-stacks and quantification (n=6) of (PS/SPS)₂₀-(Poly-1/Cy3-pLUC)₂₄-coated microneedle arrays (left-brightfield, middle-before application, right-after 24 hour application, 200 μm interval, scale-200 μm). (C) Representative confocal z-stacks and quantification (n=6) of (PS/SPS)₂₀-(Poly-1/DiI-PLGA-NP)₄ coated microneedle arrays (left-brightfield, middle-before application, right-after 5 minute application, 200 μm interval, scale-200 μm). Representative confocal micrographs (1-MHC-GFP II, 2-Cy3-pLUC, 3-DiI/D-PLGA NP, 4-overlay, scale-200 μm) showing dorsal ear skin following (D) 5 minute and (E) 24 hour application of a (PS/SPS)₂₀-(Poly-1/Cy3-pLUC)₂₄ coated microneedle array, (F) 5 minute (PS/SPS)₂₀-(Poly-1/DiI-PLGA-NP)₄ coated microneedle application, and (G) 24 hour (PS/SPS)₂₀-(Poly-1/Cy3-pLUC)₂₄-(Poly-1/DiD-PLGA NP)₄ coated microneedle application.

FIG. 14 In vivo bioluminescent signal observed in C57BL/6 mice (n=3) following treatment with a (PS/SPS)₂₀-(Poly-1/pLUC)ₙ-coated microneedle array to the right ear (denoted by arrow): (A) 24 bilayers for 5 minutes, (B) 1 bilayer for 24 hours, (C) 5 bilayers for 24 hours, and (D) 24 bilayers for 24 hours. The bioluminescent results following treatment are summarize in (E, F) for 7 days together with the negative control signal (denoted C) collected from the untreated ear, with (E) demonstrating the effect of application time and (F) showing the result of increasing pLUC dosage.

FIG. 1 Schematic of PLGA microneedle fabrication process. (A) PDMS slabs were machined using laser ablation to create micron scale cavities before (B) application of PLGA to the surface of the mold. (C) PLGA was then melted under vacuum and cooled before (D) removal from the PDMS mold. (E) Schematic showing the LbL self-assembly process of iterative deposition of oppositely charged polymers through immersion. (F) Initial multilayers were deposited using alternating immersion of PLGA microneedle arrays in solutions of polycationic protamine sulfate and polyanionic poly(4-styrene sulfate). (G) Additional multilayers were then deposited through alternating deposition of polycationic polymer-1 and polyanionic plasmid DNA or PLGA NP to give pDNA or PLGA NP coated arrays respectively. Microneedle arrays coated with both pDNA and PLGA NP were constructed in a similar way first depositing PS/SPS base layers, followed by poly-1/pDNA multilayers and finally poly-1/PLGA NP multilayers.

FIG. 3 SEM micrograph of uncoated PLGA microneedle arrays of conical geometry (scale-500 μm).

FIG. 4 Chemical structure of poly-1 used in this study (molecular weight ˜8,000-10,000 g/mol).

FIG. 5(A) representative CLSM z-stacks of a (PS/SPS)₂₀-(Poly-1/Cy3-pLUC)₂₄ coated microneedle array and (B) a (PS/SPS)₂₀-(Poly-1/DiI-PLGA NP)₄ coated microneedle array. (C) CLSM z-stack of dual coated microneedle array (PS/SPS)₂₀-(Poly-1/Cy3-pLUC)₂₄-(Poly-1/DiD-PLGA NP)₄ (scale bar-100 μm).

FIG. 7 In vivo skin penetration results for microneedle arrays with (A) pyramidal geometry and (B) conical geometry (left-optical micrographs of microneedle arrays before and after application, right-trypan blue staining of microneedle penetration patterns).

FIG. 8 In vivo skin penetration results for microneedle application to MHC II-GFP mice. CLSM images showing microneedle penetration colocalized with Langerhans DCs in the epidermis (scale bar-100 μm).

FIG. 9 Representative CLSM z-stacks of a (PS/SPS)₂₀-(Poly-1/Cy3-pLUC)₂₄ coated microneedle array (A) before application, (B) after a 5 minute application, and (C) after a 24 hour application in vivo. (D) Quantification (n=6) of relative integrated Cy3 signal on both the microneedle surface and the base of the array before and after application.

FIG. 10 In vivo delivery of Cy3-pLUC to ear skin of MHC II-GFP mice. CLSM images of MHC II-GFP ear skin following application of a (PS/SPS)₂₀-(Poly-1/Cy3-pLUC)₂₄ coated microneedle array for (A) 5 minutes and (B) 24 hours (scale bar-200 μm).

FIG. 11 Representative CLSM z-stacks of a (PS/SPS)₂₀-(Poly-1/DiI-PLGA NP)₄ coated microneedle array (A) before application, and (B) after a 5 minute application. (C) Quantification (n=6) of relative integrated DiI signal on both the microneedle surface and the base of the array before and after application.

FIG. 12 In vivo delivery of DiI-PLGA NP to ear skin of MHC II-GFP mice. CLSM images of MHC II-GFP ear skin following (PS/SPS)₂₀-(Poly-1/DiI-PLGA NP)₄ coated microneedle array application for 5 minutes indicates effective transfer of PEM films and delivery of PLGA NP to epidermal LCs (scale bar 100 μm).

FIG. 13 In vivo co-delivery of Cy3-pLUC and DiD-PLGA NP to ear skin of MHC II-GFP mice. CLSM z-stack images of MHC II-GFP ear skin following (PS/SPS)₂₀₋(Poly-1/Cy3-pLUC)₂₄-(Poly-1/DiD-PLGA NP)₄ coated microneedle array application for 24 hours indicates effective transfer of PEM films and delivery of pLUC and PLGA NP to epidermal LCs (scale bar 100 μm).

FIG. 15 Layer-by-layer assembly of (PrS/SPS)ₙ PEMs. Substrate is first submerged in the polycation solution,protamine sulfate (PrS); 21 of 32 amino acids are arginine, R. Following a rinse in deionized water, the PrS-coated substrate is then immersed in the polyanion solution,sodium (4-sulfonated poystyrene) (SPS), followed by another water rinse.

FIG. 16 Characterization of dry (PrS/SPS)ₙ PEM growth. (a) Profilometry measurements of dry film thickness. (b) UV-Vis spectroscopic analysis of (PrS/SPS)ₙ PEMs functionalized quartz showing absorbance associated with the amide bonds (200 nm) and aromatic amino acid residues (280 nm) of PrS and the characteristic SPS absorbance at 226 nm. (c) CD spectra of PrS-coated and (PrS/SPS)₂₀ quartz. (d) CD spectra of dry (PrS/SPS)ₙ PEM functionalized quartz.

FIG. 17. Surface morphology of (PrS/SPS)-PEMs surfaces. AFM images (10 μm×10 μm area) showed variation in the nanoscale topography of the surface of dry (PrS/SPS)ₙ PEMs at different thicknesses (zₘₐₓ=110 nm, 60 nm, 30 nm, 35 nm, 40 nm, 1500 nm, 1600 nm, and 1000 nm for n=20, 40, 60, 80, 100, 180, 200, and 240, respectively).

FIG. 18. AFM scratch test and roughness of (PrS/SPS)ₙ PEMs surfaces. AFM line scans (a and b) across the scratched (PrS/SPS)₄₀ and (PrS/SPS)₂₄₀ PEMs showed complete coverage of the substrate surface by the nanoscale thin films. (c) The RMS roughness values of the films were dependent on the thickness (bilayer number). (d) Magnification of lower n region of part b showing the decrease in RMS roughness as n increased from 20 to 100 bilayers.

FIG. 19. Liquid-phase chacterization of (PS/SPS)ₙ PEMs. (a) Dynamic air-water contact angle measurements of (PrS/SPS)ₙ PEMs. (b) In-situ spectroscopic ellipsometry thickness measurements of (PrS/SPS)ₙ PEM functionalized silicon. (c) Liquid-phase AFM measurements of Young's moduli obtained from hydrated (PrS/SPS)ₙ PEM functionalized silicon surfaces.

FIG. 20. MC3T3-E1 morphology on conventional tissue culture substrates (TCPS and glass) and (PrS/SPS)ₙ PEM coated glass surfaces in culture medium containing 10% FBS. Calcein deposits in the cytoplasm of MC3T3-E1 cells demonstrated alterations in cell morphology (cytoplasm area to total cell area ratio) and cytoplasm projections on PrS/SPS coated surfaces compared to TCPS and uncoated glass surface.

FIG. 21. Adhesion and proliferation of MC3T3-E1 on PEMs in serum-free and serum-containing cultures. MC3T3-E1 adhesion to (PrS/SPS)ₙ PEMs coated and uncoated glass surfaces in serum-free culture medium (a) and in culture medium containing 10% FBS (b). (c) MC3T3-E1 pre-osteoblast seeded at 5,000 cells/cm² onto (PrS/SPS)ₙ functionalized glass surfaces and to control surfaces (TCPS and uncoated glass) proliferated in presence of 10% FBS. (d) Metabolic activity of MC3T3-E1 pre-osteoblasts seeded at high-density (50,000 cells/cm²) on uncoated and (PrS/SPS)ₙ functionalized glass surfaces were determined after 1 week and 3 weeks culture.

FIG. 22. Osteogenic differentiation of MC3T3-E1 cells on (PrS/SPS)ₙ PEMs. Differentiation of MC3T3-E1 cells seeded at high density (50,000 cells/cm²) on uncoated and (PrS/SPS)ₙ functionalized glass surfaces was quantified by alkaline phosphatase activity (ALP; a) and Alizarin Red S (ARS) at 15 days (b), 22 days (c), and 27 days (d) of culture in osteogenic media.

FIG. 23. Long-term culture of MC3T3-E1 cells on (PrS/SPS)ₙ PEMs. Micrographs of MC3T3-E1 cells on uncoated (PrS/SPS)₀, (PrS/SPS)₄₀, and (PrS/SPS)₈₀ during proliferation (brightfield) and differentiation (Alizarin Red S and von Kossa). All surfaces supported adhesion and proliferation of MC3T3-E1 cells to achieve near confluence prior to onset of osteogenic differentiation. Alizarin Red S staining demonstrated increased calcium deposits with increasing thickness of the PEMs. Von Kossa staining showed that mineralization of these calcium deposits was also dependent on the PEM thickness.

FIG. 24 Real-time QCM-D measurement of SPS and PrS mass deposition during (PrS/SPS)ₙ PEM assembly. (a) Increase in D during the sequential deposition of each polyelecrolyte on the surface of the oscillating crystal. (b) Post-rinse plateau D after deposition of each polymer. (c) The AD associated with adsorbing PrS and SPS during the (PrS/SPS)ₙ PEM growth. (d) Decrease in frequency associated with increasing adsorption of polymer on crystal surface. (e) Post-rinse plateau of frequency after deposition of each polymer and mass calculated from Sauerbrey's relation. (f) The adsorbed mass associated with adsorbing PrS and SPS during the (PrS/SPS)ₙ PEM growth.

FIG. 25. AFM measurements of RMS roughness of (PrS/SPS)ₙ PEMs. (a) AFM images of (PrS/SPS)₂₀₀ and (PrS/SPS)₂₄₀ PEMs equilibrated in PrS deposition buffer and in phosphate buffer saline. (b) RMS roughness of PrS and PBS equilibrated (PrS/SPS)₂₀₀ and (PrS/SPS)₂₄₀ PEMs compared to the de novo films prepared by the LbL fabrication protocol.

### DETAILED DESCRIPTION OF CERTAIN EMBODIMENTS

In accordance with the present invention, multilayer films can be used to coat a substrate. In various embodiments, compositions and methods for a protamine polypeptide-containing multi-layer film are disclosed.

### Multilayer Film

Multilayer films provided in the present invention may have various thickness depending on methods of fabricating and applications. In some embodiments, a multilayer film has an average thickness in a range of about 1 nm and about 100 μm In some embodiments, a multilayer film has an average thickness in a range of about 300 nm and about 500 nm. In some embodiments, a multilayer film has an average thickness in a range of about 2 μm and about 5 μm In some embodiments, the average thickness of a multilayer film is or more than about 1 nm, about 100 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 10 μm, bout 20 μm, about 50 μm, or about 100 μm In some embodiments, a multilayer film has an average thickness in a range of any two values above.

In general, a multilayer film can be or comprises a plurality of units (e.g., a bilayer unit, a tetralayer unit, etc.). In some embodiments, a unit has an average thickness in a range of about 0.5 nm and about 100 nm. In some embodiments, a unit has an average thickness in a range of about 1 nm and about 5 nm. In some embodiments, a unit has an average thickness in a range of about 2 μm and about 5 μm In some embodiments, the average thickness of a unit is or more than about 0.5 nm, 1 nm, 1.1 nm, 1.2 nm, 1.3 nm, 1.4 nm, 1.5 nm, 1.6 nm, 1.7 nm, 1.8 nm, 1.9 nm, 2 nm, 2.5 nm, 3 nm, 4 nm, 5 nm, 10 nm, 50 nm, or 100 nm. In some embodiments, a unit has an average thickness in a range of any two values above.

In some embodiments, a plurality of units used in accordance with the present invention comprises a number of the unit. In some embodiments, the number of a unit can be about or more than 1, 2, 5, 10, 20, 30, 40, 50, 80, 100, 120, 150, 180, 200, 240, 300, 400, 500, 1000. In some embodiments, the number of a unit can be in a range of any two value above.

In some embodiments, a multilayer film is a composite that include more than one units. For example, more than one units can have be different in film materials (e.g., polymers), film architecture (e.g., bilayers, tetralayer, other multilayer units, etc.), film thickness, and/or releasable agents that are associate with one of the units. In some embodiments, a multilayer film is a composite that include more than one bilayer units, more than one tetralayer units, or any combination thereof. In some embodiments, a multilayer film is a composite that include a plurality of a first unit and a plurality of a second unit. In some embodiments, a multilayer film is a composite that include a plurality of a first and a second unit, and further a plurality of a unit. In some embodiments, a multilayer film comprise a single layer/unit; such a layer/unit may be inert (e.g., not associated with adjacent layers).

Multilayer films may be comprised of at least one unit comprising a layer and its adjacent layer being associated with one another by non-covalent bonding, covalent bonding or any combination thereof. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

In some embodiments, multilayer films comprising at least one unit comprising a layer and its adjacent layer being associated with one another via electrostatic interactions. For example, such multilayer films can have at least one unit with alternating layers of opposite charges, such as alternating anionic and cationic layers. In some embodiments, a multilayer film include a plurality of polyelectrolyte layers.

In some embodiments, at least one of the layers in a multilayer film includes a degradable polyelectrolyte. Degradable polyelectrolytes and their degradation byproducts may be biocompatible so as to make multilayer films amenable to use in vivo.

In some embodiments, a multilayer film comprises a protamine polypeptide.

Polyions

In accordance with the present invention, polyionic layers may be used in film construction and placed next to a layer having an opposite charge. In various embodiments, a multilayer film can comprise one or more polyions. In some embodiments, a polyionic layer is or comprises a polyanion. In some embodiments, a polyionic layer is or comprise a polycation.

Exemplary multilayer films and polyions suitable for use in accordance with the present invention are described in U.S. Pat. No. 7,112,361; U.S. Ser. No. 11/815,718, filed Oct. 29, 2008; U.S. Ser. No. 11/473,806, filed Jun. 22, 2006; U.S. Ser. No. 12/278,390, filed Aug. 5, 2008; U.S. Ser. No. 11/459,979, filed Jul. 26, 2006; U.S. Ser. No. 12/139151, filed Jun. 13, 2008; U.S. Ser. No. 12/406,369, filed Mar. 18, 2009; and U.S. Ser. No. 12/542,267, filed Aug. 17, 2009, the entire contents of each of which are incorporated herein by reference.

For example, in some embodiments, a multilayer film comprise a tetralayer unit having the structure (degradable cationic polyelectrolyte/polyanion/cationic polymeric cyclodextrin/polyanion). (Structures with reversed or modified charge schemes, e.g., comprising anionic polyelectrolytes, polycations, and anionic cyclodextrins, may also be possible.) In some embodiments, a multilayer film comprise a tetralayer unit having the structure (degradable cationic polyelectrolyte/polyanion/cationic drug layer/polyanion). (Structures with reversed or modified charge schemes, may also be possible.)

In some embodiments, polyions are not degradable, though they may be. Polyions used herein are generally biologically derived, though they need not be.

In some embodiments, a polyion used in a multilayer film disclosed herein can be a degradable polymer. Such a degradable polymer can be hydrolytically degradable, biodegradable, thermally degradable, and/or photolytically degradable polyelectrolytes.

Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. The properties of these and other polymers and methods for preparing them are further described in the art. See, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. Nos. 4,806,621; 4,638,045 to Kohn; and U.S. Pat. No. 4,946,929 to d'Amore; see also Wang et al., J. Am. Chem. Soc. 123:9480, 2001; Lim et al., J. Am. Chem. Soc. 123:2460, 2001; Langer, Acc. Chem. Res. 33:94, 2000; Langer, J. Control Release 62:7, 1999; and Uhrich et al., Chem. Rev. 99:3181, 1999. Of course, co-polymers, mixtures, and adducts of these polymers may also be employed.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself Cationic polyelectrolytes may be degradable polymers with cationic groups distributed along the polymer backbone. Cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself

For example, a range of hydrolytically degradable amine containing polyesters bearing cationic side chains have been developed (Putnam et al. Macromolecules 32:3658-3662, 1999; Barrera et al. J. Am. Chem. Soc. 115:11010-11011, 1993; Kwon et al. Macromolecules 22:3250-3255, 1989; Lim et al. J. Am. Chem. Soc. 121:5633-5639, 1999; Zhou et al. Macromolecules 23:3399-3406, 1990; each of which is incorporated herein by reference). Examples of these polyesters include poly(L-lactide-co-L-lysine) (Barrera et al. J. Am. Chem. Soc. 115:11010-11011, 1993; incorporated herein by reference), poly(serine ester) (Zhou et al. Macromolecules 23:3399-3406, 1990; which is incorporated herein by reference), poly(4-hydroxy-L-proline ester) (Putnam et al. Macromolecules 32:3658-3662, 1999; Lim et al. J. Am. Chem. Soc. 121:5633-5639, 1999; each of which is incorporated herein by reference), and more recently, poly[α-(4-aminobutyl)-L-glycolic acid].

Poly(β-Amino Ester)

Poly(β-amino esters) prepared from the conjugate addition of primary or secondary amines to diacrylates, are suitable for use in accordance with the present invention. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, a polymer used in accordance with the present invention can have a formula below:

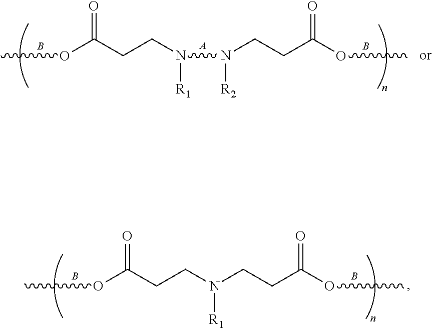

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

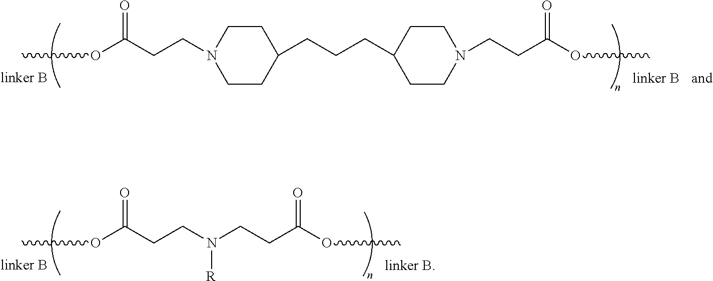

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units.

In some embodiments, poly(β-amino ester)s are selected from the group consisting of

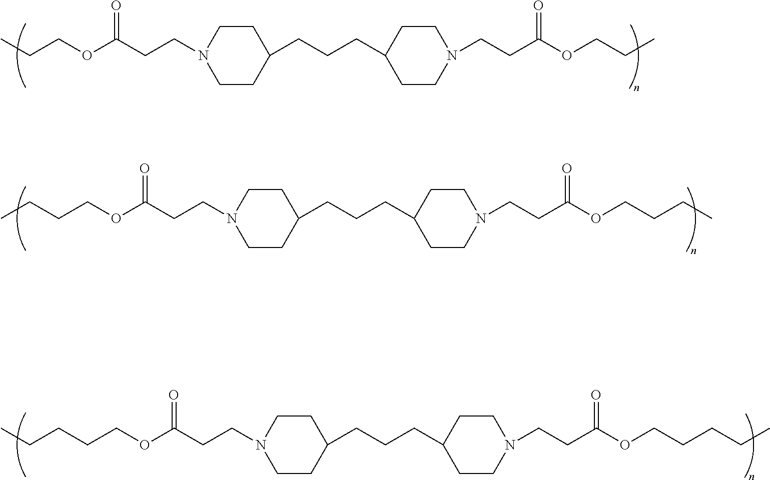

derivatives thereof, and combinations thereof.

Protamine Polypeptide

Protamine polypeptides may be suitable for use in accordance with the present invention. Typically, a protamine polypeptide is or comprises a short proteins (50-110 amino acids) that can contain up to 70% arginine. In some embodiments, a multilayer film in accordance with the present invention includes a protamine polypeptide. In some embodiments, a multilayer film is or comprises a plurality of units, each unit containing a protamine polypeptide.

Without being bound to any particular theory, such multilayer films containing a protamine polypeptide are particularly useful for gene delivery. In some embodiments, such multilayer films containing a protamine polypeptide and a therapeutic gene. For example, a multilayer film containing a protamine polypeptide and a DNA can be constructed via a hybrid mechanism including electrostatic interactions between polyelectrolyte layers as well as allosteric interactions between proamine polypeptides, between DNAs, and/or between protamine polypeptide-DNA.

In some embodiments, a salt form of a protamine polypeptide is used in accordance with the present invention. For example, a salt form of a protamine polypeptide can be protamine sulfate, which is a natural polyamine polypeptide that facilitates condensation of DNA in sperm and plays a pivotal role during fertilization.

Alternatively or additionally, charged polysaccharides may be used as a polyion in constructing a multilayer film. In some embodiments, polysaccharides include glycosaminoglycans such as heparin, chondroitin, dermatan, hyaluronic acid, etc. (Some of these terms for glycoasminoglycans are often used interchangeably with the name of a sulfate form, e.g., heparan sulfate, chondroitin sulfate, etc. It is intended that such sulfate forms are included among a list of exemplary polyions used in accordance with the present invention. Similarly, other derivatives or forms of such polysaccharides may be incorporated into films.)

Polyions that may be used in accordance with the present invention include zwitterionic polyelectrolytes. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, a multilayer film may be constructed by LbL deposition using dip coating in solutions of a first pH at which one layer is anionic and a second layer is cationic. If such a multilayer film is put into a solution having a second different pH, then the first layer may be rendered cationic while the second layer is rendered anionic, thereby changing the charges on those layers.

In some embodiments, polyions alter or tune characteristics of a multilayer film that are useful for medical applications. For example, the degradation rate of a multilayer film can be adjusted by combining with a degradable polyeletrolyte as discussed above. Polyions may also interact or impart a layer comprising a releasable agent to be released, and thus adjust the release rate/kinetics of the releasable agent.

### Releasable Agents

According to the present invention, multilayer films can include one or more releasable agents for delivery. In some embodiments, a multilayer film includes more than one units and one or more releasable agents. In some embodiments, a releasable agent can be associated with individual layers of a multilayer film for incorporation, affording the opportunity for exquisite control of loading and release from the film. In certain embodiments, a releasable agent is incorporated into a multilayer film by serving as a layer.

A wide range of agents may be incorporated within a multilayer film for delivery with the provided films and/or structures. In general, a releasable agent may include, but are not limited to, any therapeutic agents (e.g. antibiotics, NSAIDs, glaucoma medications, angiogenesis inhibitors, neuroprotective agents), cytotoxic agents, diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.), or other substance that may be suitable for introduction to biological tissues, including pharmaceutical excipients and substances for tattooing, cosmetics, and the like.

In some embodiments, a releasable agent can be a substance having biological activity. In some embodiments, a releasable agent may be or comprise small molecules, large (i.e., macro-) molecules, or a combination thereof In some embodiments, an agent can be a drug formulation including various forms, such as liquids, liquid solutions, gels, hydrogels, solid particles (e.g., microparticles, nanoparticles), or combinations thereof

In representative, non-limiting, embodiments, a releasable agent can be selected from among amino acids, vaccines, antiviral agents, nucleic acids (e.g., siRNA, RNAi, and microRNA agents), gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, vitamins and/or any combination thereof In some embodiments, an releasable agent may be selected from suitable proteins, peptides and fragments thereof, which can be naturally occurring, synthesized or recombinantly produced.

In some embodiments, a releasable agent can be growth factors such as osteogenic factors. For example, a multilayer film comprising an osteogenic factor can greatly enhance the rate and extent of mineralization at a tissue repair site being contacted with a coated substrate (e.g., an implant).

In some embodiments, compositions and methods in accordance with the present invention are particularly useful for release of one or more therapeutic agents.

In some embodiments, a therapeutic agent is a small molecule and/or organic compound with pharmaceutical activity. In some embodiments, a therapeutic agent is a clinically-used drug. In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, 3-adrenergic blocking agent, diuretic, cardiovascular active agent, vasoactive agent, anti-glaucoma agent, neuroprotectant, angiogenesis inhibitor, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent for release used in accordance with the present invention is an agent useful in combating inflammation and/or infection.

In some embodiments, a therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof

An antibiotic may be bacteriocidial or bacteriostatic. Other anti-microbial agents may also be used in accordance with the present invention. For example, anti-viral agents, anti-protazoal agents, anti-parasitic agents, etc. may be of use.

In some embodiments, a therapeutic agent may be an anti-inflammatory agent. Anti-inflammatory agents may include corticosteroids (e.g., glucocorticoids), cycloplegics, non-steroidal anti-inflammatory drusg (NSAIDs), immune selective anti-inflammatory derivatives (ImSAIDs), and any combination thereof Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof

A variety of agents known in the art may be formulated for administration. Examples include antagonists (e.g., carteolol, cetamolol, betaxolol, levobunolol, metipranolol, timolol), miotics (e.g., pilocarpine, carbachol, physostigmine), sympathomimetics (e.g., adrenaline, dipivefrine), carbonic anhydrase inhibitors (e.g., acetazolamide, dorzolamide), prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), anti-viral compounds (e.g., acyclovir, cidofovir, idoxuridine, interferons), aldose reductase inhibitors, anti-inflammatory and/or anti-allergy compounds (e.g., steroidal compounds such as betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors.

In some embodiments, a therapeutic agent may a therapeutic gene as known in the art. In some embodiments, a therapeutic agent is a non-viral vector. Typical non-viral gene delivery vectors comprise DNA (e.g, plasmid DNA produced in bacteria) or RNA. In certain embodiments, a non-viral vectors is used in accordance with the present invention with the aid of a delivery vehicle. Delivery vehicles may be based around lipids (e.g, liposomes) which fuse with cell membranes releasing a nucleic acid into the cytoplasm of the cell. Alternatively or alternatively, peptides or polymers may be used to form complexes (e.g., in form of paritices) with a nucleic acid which may condense as well as protect the therapeutic activity as it attempts to reach a target destination.

In some embodiment, a releasable agent in accordance with the present invention is in form of particles. In theory, particles can be of any shape or size. For example, nanoparticles and/or microparticles may have a dimension in a range of 1 to 100 μm to 25 μm or 1 to 1000 nm. Exemplary particles that may be incorporated within a multilayer film include solid or gel-like organic or inorganic compounds in a non-dissolving solvent (e.g., barium sulfate suspension in water), liposomes, proteins, cells, virus particles, prions, and combinations thereof In some embodiments, at least one therapeutic agent is incorporated into a particle.

### Substrates

A substrate may be coated with one or more multilayer films in accordance with the present invention. A variety of entities or materials can be used as a substrate for constructing multilayer films. Exemplary entities or materials include, but are not limited to, metals (e.g., gold, silver, platinum, and aluminum); metal-coated materials; metal oxides; plastics; ceramics; silicon; glasses; mica; graphite; hydrogels; and polymers such as polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof In some embodiments, a substrate may comprise more than one material to form a composite.

In some embodiments, a substrate is or comprises a medical device. In some embodiments, a medical device is an implant. Exemplary medical implants include, for example, catheters (e.g., vascular and dialysis catheters), heart valves, cardiac pacemakers, implantable cardioverter defibrillators, grafts (e.g., vascular grafts), ear, nose, or throat implants, urological implants, endotracheal or tracheostomy tubes, CNS shunts, orthopedic implants, and ocular implants.

Microneedle Substrates

Microneedle substrates, for example, can be used in accordance with the present invention. Coated microneedle substrates and methods for coating are described herein, enabling various multilayer films containing agents to be controllably coated onto microneedle substrates. Such coated microneedle substrates can be contacted with biological tissues, particularly for transdermal delivery of agents.

In some embodiments, a microneedle substrate is provided which includes at least one microneedle having a base, a tip end, and a shaft portion therebetween, and a multilayer film coating on at least a portion of the surface of the microneedle. In some embodiments, the multilayer film coating includes at least one releasable agents. Such multilayer film coatings can be a homogeneous or a heterogeneous composition.

A microneedle substrate can be formed/constructed of different biocompatible materials, including metals, glasses, semi-conductor materials, ceramics, or polymers. Examples of suitable metals include pharmaceutical grade stainless steel, gold, titanium, nickel, iron, tin, chromium, copper, and alloys thereof In some embodiments, stainless steel is an attractive material for microneedle fabrication because it is FDA approved for medical devices and is inexpensive.

In some embodiments, a microneedle substrate may include or be formed of a polymer. A polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof Representative non-biodegradable polymers include polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof Biodegradable microneedles can provide an increased level of safety compared to non-biodegradable ones, such that they are essentially harmless even if inadvertently broken off into the biological tissue being contacted with.

In some embodiments, a microneedle substrate includes a substantially planar foundation from which one or more microneedles extend, typically in a direction normal (i.e., perpendicular or ‘out-of-plane’) to the foundation. Additionally or alternatively, microneedles may be fabricated on the edge of a substrate ‘in-plane’ with the substrate. In some embodiments, a single microneedle can be fabricated on a substrate surface or edge. In some embodiments, microneedles are fabricated on a flexible base substrate. It would be advantageous in some circumstances to have a base substrate that can bend to conform to the shape of the surface of a biological tissue being contacted with. In some embodiments, the microneedles are fabricated on a curved base substrate. The curvature of the base substrate typically would be designed to conform to the shape of the tissue surface.

Microneedles in theory can be of any shape or design. A microneedle may be solid or hollow. A microneedle can be porous or non-porous. A microneedles may be planar, cylindrical, or conical.

In some embodiments, the dimensions of a microneedle, or array thereof, are designed for the particular way in which it is to be used. In various embodiments, the microneedle may have a dimention in a range of between about 50 μm and about 5000 μm, about 100 μm and about 1500 μm, or between about 200 μm and about 1000 μm.

In some embodiments, a microneedle substrate includes a single microneedle or an array of two or more microneedles. The microneedles can be fabricated as, or combined to form microneedle arrays. For example, a microneedle substrate may include an array of between 2 and 1000 (e.g., between 2 and 100) microneedles. In some embodiments, a microneedle substrate may include an array of between 2 and 10 microneedles. An array of microneedles may include a mixture of different microneedles. For instance, an array may include microneedles having various lengths, base portion diameters, tip portion shapes, spacings between microneedles, drug coatings, etc.

### Assembly and Coating Methods

There are several advantages to LBL assembly techniques used in accordance with the present invention, including mild aqueous processing conditions (which may allow preservation of biomolecule function); nanometer-scale conformal coating of surfaces; and the flexibility to coat objects of any size, shape or surface chemistry, leading to versatility in design options. According to the present invention, one or more multilayer films can be assembled and/or deposited on a substrate using a LBL technique. The coating compositions and methods provided herein may be used for coating a substrate (e.g., microneedle substrates). In various embodiments, one or more multilayer films can be the same. In some embodiments, one or more multilayer films can be different in film materials (e.g., polymers), film architecture (e.g., bilayers, tetralayer, etc.), film thickness, and/or agent association.

It will be appreciated that an inherently charged surface of a substrate can facilitate LbL assembly of a multilayer film on the substrate. In addition, a range of methods are known in the art that can be used to charge the surface of a substrate, including but not limited to plasma processing, corona processing, flame processing, and chemical processing, e.g., etching, micro-contact printing, and chemical modification.

In some embodiments, a substrate is first coated with a precoat material. Such a precoat can be used to alter or improve the surface properties (e.g., hydrophilicity or hydrophobicity) of the substrate surface to enhance adhesion and uniformity of multilayer film coatings. A precoat may be substantially soluble or insoluble in vivo. In non-limiting examples, a precoat may consist of silicon dioxide or a biocompatible polyester, polyethylene glycol (PEG), PLGA or polyanhydride. Deposition of silicon dioxide or other precoat material may be achieved using vapor deposition or other techniques known in the art.

Additionally or alternatively, an exterior, secondary coating may be used to alter release kinetics of an agent from an underlying coating layer. For example, an exterior coating may include a material known in the art that dissolves or biodegrades relatively solely in vivo to provide delayed or slow release of drug. In one example, an exterior coating could include a hydrogel or other water swellable material to provide controlled agent release. In another variation, an exterior layer could provide for rapid (e.g., bolus) release of an agent. An underlying layer could provide bolus or controlled release of the same or another agent.

In some embodiments, substrates can be primed with specific polyelectrolyte bilayers such as, but not limited to, LPEI/SPS, PDAC/SPS, PAH/SPS, LPEI/PAA, PDAC/PAA, and PAH/PAA bilayers, that form readily on weakly charged surfaces and occasionally on neutral surfaces. Exemplary polymers can be used as a primer layer include poly(styrene sulfonate) and poly(acrylic acid) and a polymer selected from linear poly(ethylene imine), poly(diallyl dimethyl ammonium chloride), and poly(allylamine hydrochloride). It will be appreciated that primer layers provide a uniform surface layer for further LBL assembly and are therefore particularly well suited to applications that require the deposition of a uniform thin film on a substrate that includes a range of materials on its surface, e.g., an implant or a complex tissue engineering construct.

In some embodiments, the LbL assembly of a multilayer film may involve a series of dip coating steps in which a substrate is dipped in alternating polycationic and polyanionic solutions. Additionally or alternatively, it will be appreciated that deposition of alternating polycationic and polyanionic layers may also be achieved by spray coating, dip coating, brush coating, roll coating, spin casting, or combinations of any of these techniques.

Methods described herein provide for coatings may be particularly useful for microneedle substrates. In some embodiments, a coated structure includes a microneedle substrate and a multilayer film coating that contains or consists of at least one releasable agent. Such a coated structure, for example, may be incorporated into a transdermal drug delivery patch or other drug delivery device.

Those of ordinary skill in the art will appreciate that there are a variety of substrates as described above can be coated by the provided methods in the present invention. In addition to microneedle substrates, it also is envisioned that the present coating methods and devices can be used or readily adapted to coat other microstructures, particularly structures having micron-scale dimensions where surface tension issues impact coating location, coating thickness, and coating processibility. Representative examples of other microstructures include microfluidic devices, microarrays, microelectrodes, AFM probes, microporous materials, microactuators, microsensors, and the like.

### Use and Applications

Compositions and methods provide herein can be of use various application such as coating substrate (e.g., microneedle substrates) using a multi-layer film assembled LBL. Also provided in the disclosure are methods of releasing one or more releasable agents from a multilayer film.

In general, multilayer films may be exposed to a medium (e.g., intracellular fluid, interstitial fluid, blood, intravitreal fluid, intraocular fluid, gastric fluids, etc.). In some embodiments, a medium can be provided in an artificial environment (e.g., for tissue engineering scaffolds). Buffers such as phosphate-buffered saline may also serve as a suitable medium.

In some embodiments, provided methods herein comprise steps of providing a multilayer film and placing the film in a medium in which at least a portion of the film decomposes via the substantially sequential removal of at least a portion of the layers having the first charge and degradation of layers having the second charge. Releasable agents are thus gradually and controllably released from the multilayer film. It will be appreciated that the roles of the layers of a multilayer film can be reversed.

Certain characteristics of a multilayer film-coated substrate may be modulated to achieve desired doses and/or release kinetics of releasable agents.

For example, degradation of a multilayer film in accordance with the present invention can be fine-tuned by adjusting the composition of the film. In some embodiments, the degradation rate of each layer within a multilayer film can be adjusted, which is believe to impact the release rate of drugs. In some embodiments, the degradation rate of a hydrolytically degradable polyelectrolyte layer can be decreased by associating hydrophobic polymers such as hydrocarbons and lipids with one or more of the layers. Alternatively, polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions. In other embodiments, polyanionic and/or polycationic layers may include a mixture of degradable and non-degradable polyelectrolytes. Any non-degradable polyelectrolyte can be used. Exemplary non-degradable polyelectrolytes that could be used in multilayer films include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Alternatively or additionally, the degradation rate may be fine-tuned by associating or mixing non-biodegradable, yet biocompatible polymers (polyionic or non-polyionic) with one or more of the polyanionic and/or polycationic layers. Suitable non-biodegradable, yet biocompatible polymers are well known in the art and include polystyrenes, certain polyesters, non-biodegradable polyurethanes, polyureas, poly(ethylene vinyl acetate), polypropylene, polymethacrylate, polyethylene, polycarbonates, and poly(ethylene oxide)s.

Doses of a releasable drug in accordance with the present invention may be modulated, for example, by changing the number of multilayer units that make up the film, the type of degradable polyelectrolyte used, the type of other polyion used, and/or concentrations of solutions of releasable agents used during construction of the films. Similarly, release kinetics (both rate of release and duration of release of an agent) may be modulated by changing any or a combination of the aforementioned factors.

In some embodiments, the dose of a releasable agent incorporated in a multilayer film for release can be about or greater than 1 mg/cm². In some embodiments, the dose of a releasable agent incorporated in a multilayer film can be about or less than 100 μg/cm². In some embodiments, the dose of a releasable agent incorporated in a multilayer film can be about or less than 50 μg/cm². In some embodiments, the dose of a releasable agent incorporated in a multilayer film can be about 10 mg/cm², about 1 mg/cm², 500 μg/cm², about 200 μg/cm², about 100 μg/cm², about 50 μg/cm², about 40 μg/cm², about 30 μg/cm², about 20 μg/cm², about 10 μg/cm², about 5 μg/cm², about 1 μg/cm², about 0.5 μg/cm², or about 0.1 μg/cm². In some embodiments, the dose of a releasable agent incorporated in a multilayer film can be in a range of any two values above.

In some embodiments, the dose per each unit of a releasable agent incorporated in a multilayer film for release can be about or greater than 1 mg/cm²/unit, independent of the thickness of the multilayer film. In some embodiments, the dose of a releasable agent incorporated in a multilayer film can be about or less than 100 μg/cm²/unit. In some embodiments, the dose of a releasable agent incorporated in a multilayer film can be about or less than 50 μg/cm²/unit. In some embodiments, the dose of a releasable agent incorporated in a multilayer film can be about 10 mg/cm²/unit, about 1 mg/cm²/unit, 500 μg/cm²/unit, about 200 μg/cm²/unit, about 100 μg/cm²/unit, about 50 μg/cm²/unit, about 40 μg/cm²/unit, about 30 μg/cm²/unit, about 20 μg/cm²/unit, about 10 μg/cm²/unit, about 5 μg/cm²/unit, about 1 μg/cm²/unit, about 0.5 μg/cm²/unit, or about 0.1 μg/cm²/unit. In some embodiments, the dose of a releasable agent incorporated in a multilayer film can be in a range of any two values above.

Release of a releasable agent may follow linear kinetics over a period of time. Release of multiple drugs from a multilayer film may be complicated by interactions between layers, and/or drugs. Such a release profile may be desirable to effect a particular dosing regimen. During all or a part of the time period of release, release may follow approximately linear kinetics.

Some embodiments provide systems for releasing a releasable agent over a period of at least about 2 hour, about 5 hours, about 12 hours, about 1 day, about 2 days, about 5 days, about 10 days, about 12 days, about 20 days, about 30 days, 50 or about 100 days. In some embodiments, a releasable agent can be released in a controlled manner over a period of any two values above.

Alternatively or additionally, a layer of cells can be deposited onto a coated structure in accordance with the present invention. Exemplary cells include, but are not limited to, connective tissue cells, organ cells, muscle cells, nerve cells, stem cells, cancer cells, and any combination thereof. In certain embodiments, cells are osteoblastic or pre-osteoblastic cells.

Without being bound to any particular theory, multilayer films comprising a protamine polypeptide and provided structures coated with such multilayer films are particularly useful for improving cellular interaction with the films and/or the structures. It is recognized in the present invention that multilayer films comprising a protamine polypeptide provide robust adhesion, proliferation, and differentiation of cells that are deposited onto the film. In some embodiments, multilayer films comprising a protamine polypeptide are characterized by enhanced stiffness and moderate swellness in aqueous environment.

### EXAMPLES

### Example 1

PLGA Microneedle Fabrication: PDMS molds (Sylgard 184, Dow Corning) were fabricated by laser ablation using a Clark-MXR, CPA-2010 micromachining system. PLGA pellets (50:50 wt lactide: glycolide, 46 kDa, Lakeshore Biomaterials) were melted over the molds under vacuum (−25 in. Hg) at 145° C. for 40 minutes, and then cooled at −20° C. before separating the cast microneedle arrays. Arrays were characterized using a JEOL 6700F FEG-SEM.

PLGA Nanoparticle Preparation: PLGA nanoparticles were prepared as previously described. Briefly, PLGA (30 mg), DOPC/DOPG lipids (4:1 mol ratio, 5 mg, Avanti Polar Lipids), and DiI or DiD (6.4 ng, Invitrogen) were co-dissolved in 1 mL dichloromethane. PBS (200 μL) was added, the emulsion was sonicated (7W, 1 min) using a Microson cell disruptor, added to 4mL of Milli-Q (MQ) water, and sonicated again (12 W, 5 min), followed by incubation for 12 hrs at 25° C. The resulting particles were purified on a sucrose gradient and analyzed using a BIC 90+ light scattering instrument (Brookhaven Instruments Corp).

Polymer Multilayer Film Preparation: All LbL films were assembled using a Carl Ziess HMS DS50 slide stainer. Films were constructed on silicon wafers, quartz slides, and PLGA microneedle arrays following treatment with O₂ plasma. To build (PS/SPS) baselayers, substrates were dipped alternatively into PS (2 mg/mL, 100 M NaOAc, Sigma-Aldrich) and SPS (5 mM, 20 mM NaCl, Sigma-Aldrich) solutions for 10 min separated by two sequential 1 minute rinses in MQ water. (Poly-1/pLUC) multilayers were deposited similarly, alternating 5 min dips in Poly-1 (2 mg/mL in 100 mM NaOAc, synthesized according to previous literature) and pLUC (1 mg/mL, 100 mM NaOAc, a gift from Dr. Daniel Barouch, Beth Israel Deaconess Medical Center) solutions separated by two sequential rinsing steps in 100 mM NaOAc, pH 5.0. Fluorescent pLUC was prepared using Cy3 Label-IT reagent (Mirus Bio Corporation). All solutions (except pLUC) were adjusted to pH 5.0 and filtered (0.2 μm) prior to dipping.

Particle Multilayer Film Preparation: Films were assembled using a previously described spray LbL technique. Briefly, microneedle arrays were coated with atomized spray solutions using modified air-brushes. Poly-1 (2 mg/mL, 100 mM NaOAc) and PLGA NP (20 mg/mL in MQ water) solutions were sprayed alternatively for 3 seconds (0.2 mL/s, 15 cm range) separated by 6 second rinses with 100 mM NaOAc. Film thickness was measured using a Tencor P-16 surface profilometer. Film delivery was characterized through CLSM imaging of microneedle arrays using a Zeiss LSM 510 and data analysis using Image J.

In Vivo Transcutaneous Delivery: Animals were cared for in the USDA-inspected MIT Animal Facility under federal, state, local, and NIH guidelines for animal care. Microneedle application experiments were performed on anesthetized C57BL/6 mice (Jackson Laboratories) and MHC II-GFP transgenic mice (a gift from Prof. Hidde Ploegh). Ears were rinsed briefly with PBS on the dorsal side and dried before application of microneedle arrays by gentle pressure. Microneedles were then removed or secured in place using Nexcare medical tape (3M). Mice were sacrificed and excised ears were stained with trypan blue before imaging for needle penetration. Ears collected from mice treated with Cy3-pLUC- and/or DiI-PLGA-NP-coated microneedle arrays were mounted on glass slides and imaged by CLSM. Transfection in mice treated with pLUC-coated arrays was measured using an IVIS Spectrum 200 (Caliper Lifesciences) to detect bioluminescence, following IP injection of luciferin.

We show here that microneedle arrays coated with DNA-carrying PEMs allows this concept to be translated to in vivo transfection in murine skin, an approach of great interest for DNA vaccine delivery. Similarly, we show that biodegradable poly(lactide-co-glycolide) (PLGA) nanoparticles (NPs), ubiquitous in drug delivery, can be embedded within microneedle PEM coatings, and subsequently deposited in the epidermis following a brief application of microneedles to unmanipulated skin. Finally, we show that multilayers combining these two diverse types of therapeutic cargos can be prepared for co-delivery into skin.

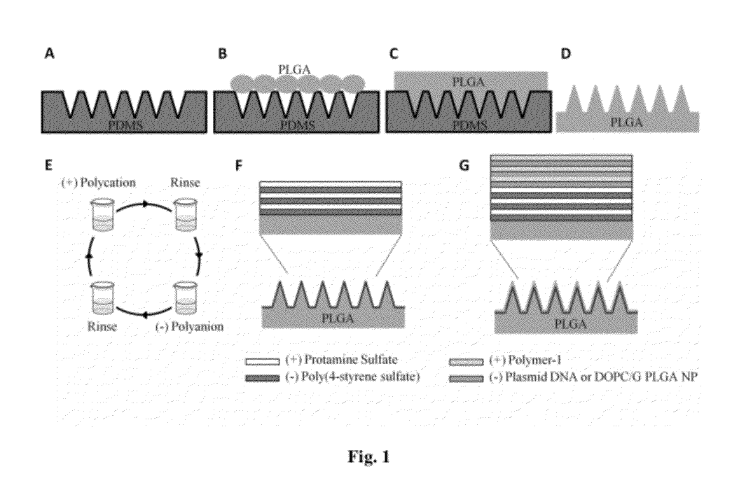

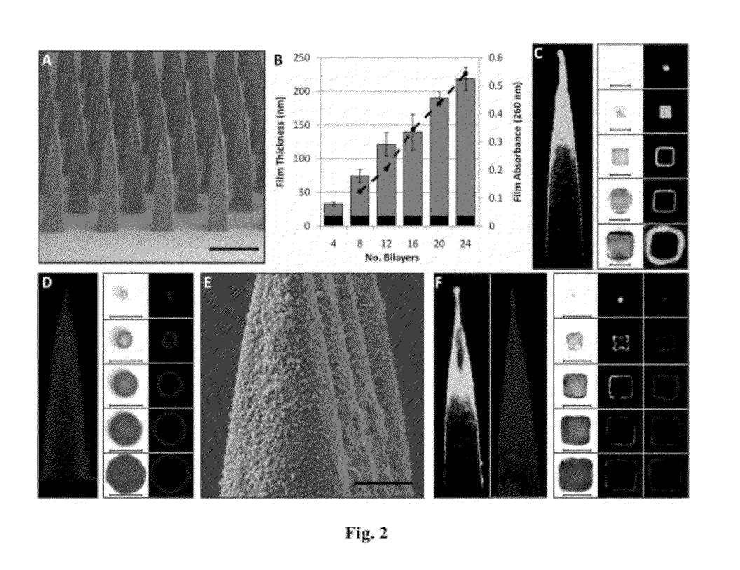

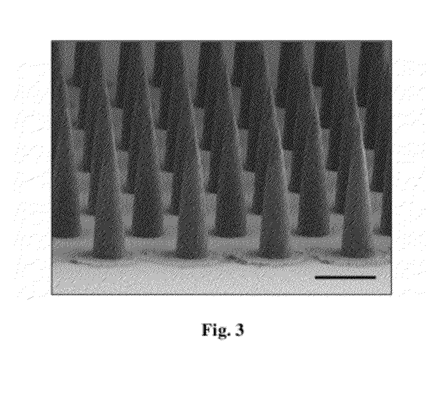

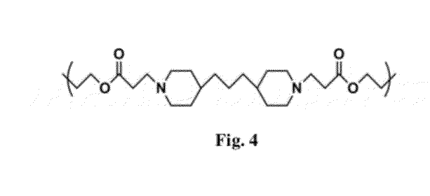

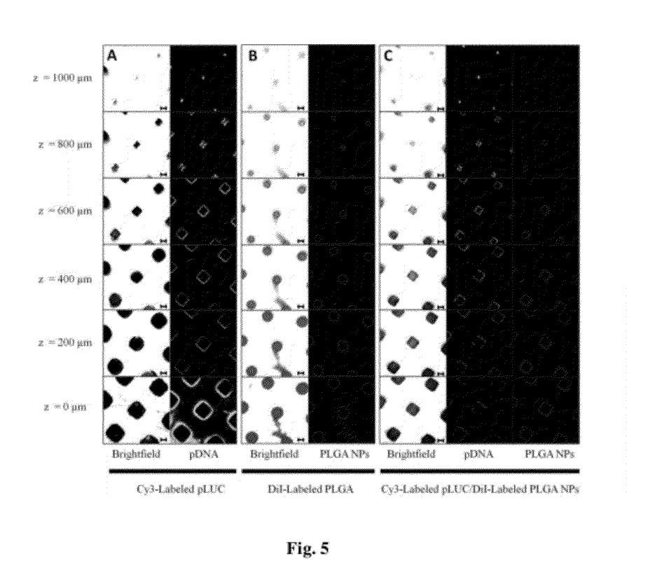

We first used laser micromachining to prepare poly(dimethylsiloxane) (PDMS) slabs with arrays of tapered pyramidal or conical microscale cavities across their surface, to serve as molds for polymer microneedle fabrication. Similar to prior reports, PLGA pellets placed over the molds were melted under vacuum, cooled, and separated from the PDMS (FIG. 1) to obtain arrays of microneedles each 250 μm in diameter at their base and 900 μm in height (FIG. 2A, FIG. 3). Microneedles of similar dimensions have been shown to produce negligible pain sensations in humans, while maintaining adequate structural integrity to efficiently penetrate the SC. To fabricate a biodegradable PEM coating capable of controlled DNA release in vivo, we employed a hydrolytically degradable poly(β3-amino ester) (PBAE), designated polymer-1(poly-1, FIG. 4). PBAEs have been previously shown to be biocompatible and degradable, to build multilayers with DNA that transfect cells in vitro, and to have adjuvant activity when co-delivered with DNA vaccines. Poly-1 in particular has been used recently by our group to fabricate LbL films with controlled erosion and tunable drug release, and by others to fabricate DNA-releasing PEM films for potential gene delivery applications. To provide a uniform initial surface charge density for PEM film growth on the PLGA microneedles, we first deposited twenty bilayers of poly(4-styrene sulfonate) (SPS), a synthetic polyanion, and protamine sulfate (PS), a mixture of four related biocompatible, highly cationic polypeptides of approximately 30 amino acids (FIG. 1). Onto this base film, PEMs were built through the alternating adsorption of poly-1 and plasmid DNA (encoding firefly luciferase, pLUC). Surface profilometry and UV absorbance indicated linear growth of (poly-1/plasmid DNA) multilayers (˜0.5±0.1 μg pDNA/cm²/bilayer) when deposited onto the (PS/SPS) base-layer (FIG. 2B). Confocal laser scanning microscopy (CLSM) was used to qualitatively examine microneedles coated with Cy3-labeled pDNA PEMs. Microneedle arrays coated in this way showed surface-localized fluorescence conformally coating each microneedle (FIGS. 2C and 5A), while control uncoated needles showed no background fluorescence (data not shown).

We next tested whether a similar approach could be used to incorporate biodegradable polymer NPs into microneedle coatings. Lipid-coated PLGA NPs (244 nm in diameter, PDI 0.15) bearing a phospholipid surface layer composed of the zwitterionic lipid DOPC, the anionic lipid DOPG, and containing a lipophilic tracer dye (DiI or DiD) were prepared using an emulsion/solvent evaporation process we recently described. Microneedles were primed with a (PS/SPS) base layer as before, and then alternating layers of poly-1 and PLGA NPs were deposited onto the arrays via spray LbL multilayer self-assembly (FIG. 1). CLSM (FIGS. 2D and 5B) and SEM (FIG. 2E) imaging of the nanoparticle PEM-coated arrays revealed conformal coatings on the microneedles, similar to the results seen with (poly-1/DNA) films. Four (poly-1/NP) bilayers produced a coating approximately 2 μm thick as measured by profilometry. In addition, serial deposition of (poly-1/pLUC) followed by (poly-1/NP) bilayers on the same microneedle array permitted the creation of films carrying both functional components (FIGS. 2F and 5C). Thus, PEM-coated microneedles have the potential to act as multifunctional delivery platforms, carrying cargos with diverse physical properties.

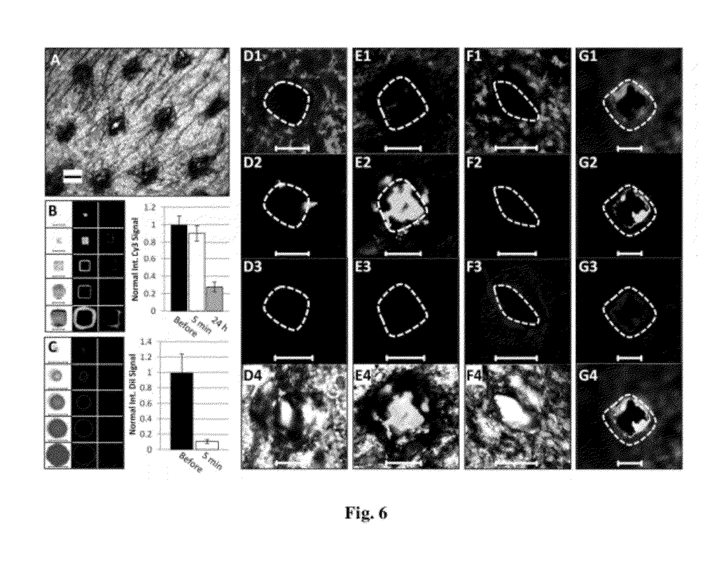

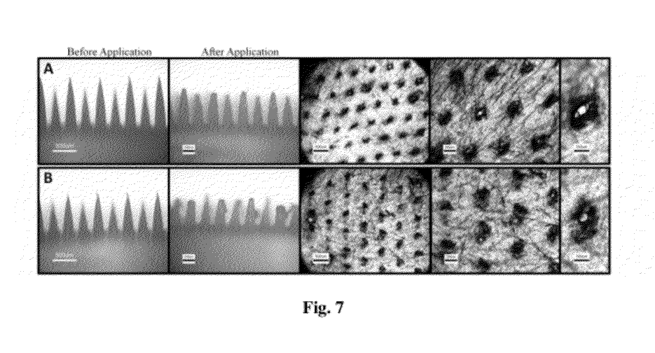

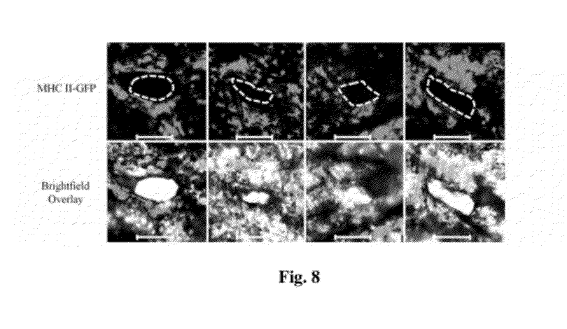

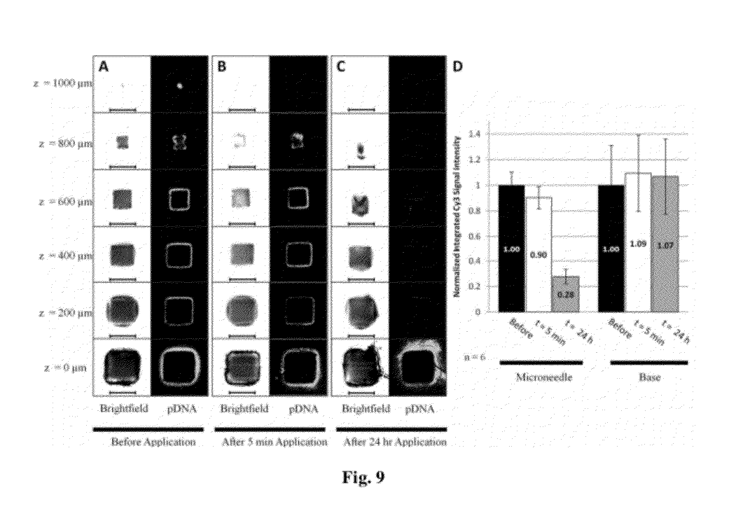

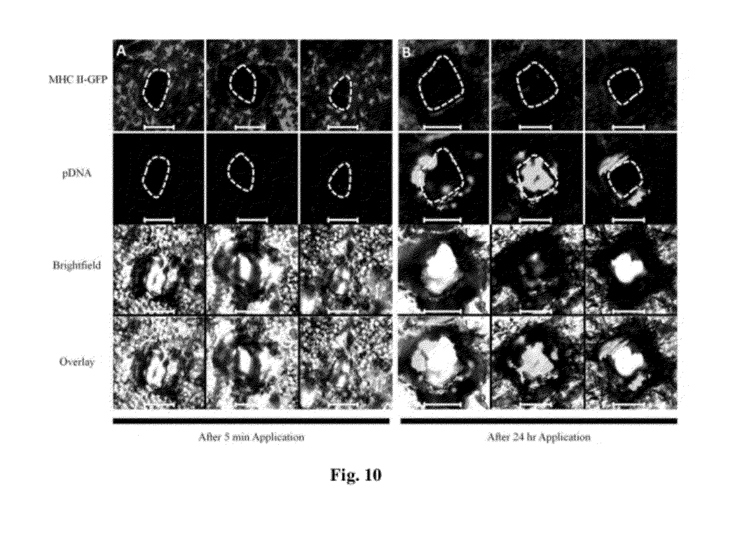

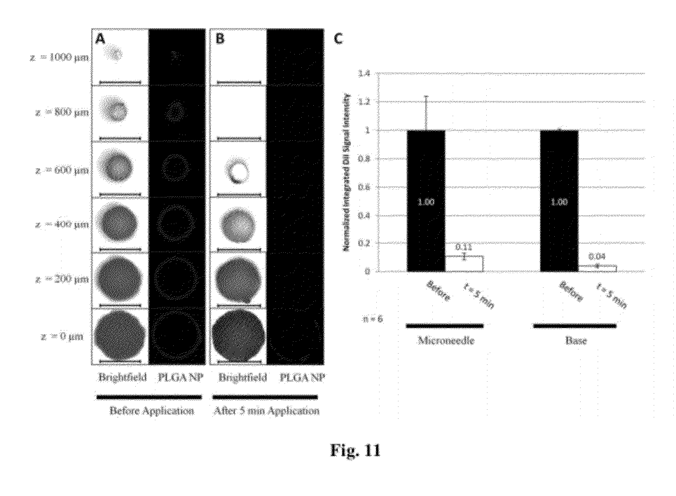

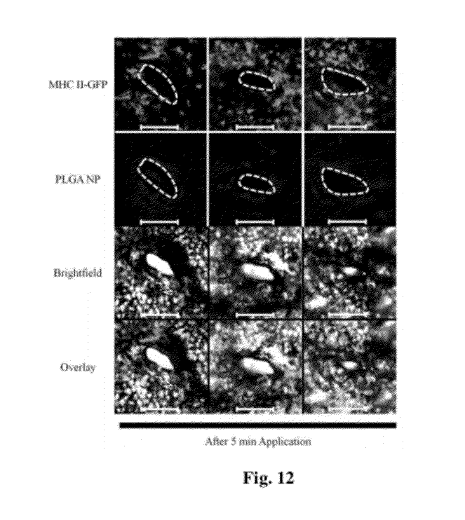

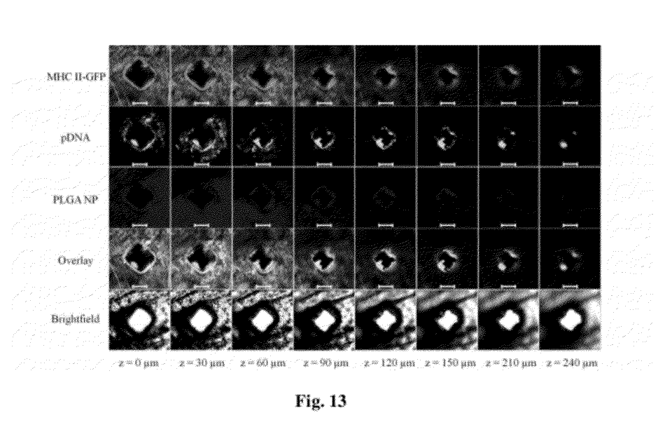

We next analyzed the penetration of microneedle arrays into the dorsal ear skin of C57B1/6 mice or C57B1/6-MHC II-GFP mice, transgenic animals expressing green fluorescent protein (GFP) fused to all class II major histocompatibility complex (MHC) molecules. The MHC II-GFP fusion protein provided an in situ fluorescence marker for the viable epidermis in skin samples from these mice, as fluorescent epidermal MHC II⁺ Langerhans cells (LCs) are readily detected by CLSM in mouse auricular skin. Prior reports have demonstrated that microneedles prepared from biodegradable polymers with suitable elastic moduli and needle geometries can penetrate human cadaver skin. To confirm that our PLGA arrays could similarly penetrate murine skin, uncoated microneedles were applied to dorsal ear skin. Trypan blue staining revealed efficient and consistent penetration of PLGA microneedle arrays through the SC; light microscopic inspection of arrays before/after application showed some buckling/bending but little breakage of the needle tips (FIGS. 6A and 7). CLSM imaging of ear skin from MHC II-GFP transgenic mice showed that microneedles readily penetrated into the viable epidermis where LCs were colocalized within the same z-plane (FIG. 8). To determine if PEM-coated microneedle arrays could deliver pDNA and/or NP cargos into the skin, we prepared PEM-coated microneedles carrying Cy3-labeled pLUC DNA (Cy3-pLUC) or DiI-labeled PLGA NPs (DiI-PLGA NPs). These PEM-coated arrays were applied to the ears of live anesthesized MHC II-GFP mice for 1 min, 5 min or 24 hrs, and then both the freshly explanted ear skin and the applied microneedles were examined by CLSM. Interestingly, the cargo delivery properties of these two types of microneedle coatings were quite distinct. Microneedles carrying (poly-1/pDNA) films examined before and after application to skin showed very little loss of DNA from the needle surfaces after applications of 1 or 5 min (FIG. 6B and FIGS. 9A, B, D, and data not shown), and little detectable transferred DNA in the epidermis (FIG. 6D and FIG. 10A), but arrays applied to skin for 24 hours led to nearly complete loss of pDNA from the microneedles (FIG. 6B and FIG. 9C, D) with a corresponding pronounced accumulation of DNA in the skin at depths colocalizing with LCs (FIG. 6E and FIG. 10B). In contrast, microneedles carrying 4 bilayers of spray-deposited (poly-1/PLGA NP) films showed immediate transfer of NPs into the epidermis and coincident loss of NP signal from the microneedles themselves following even a 5 minute application on the skin (FIGS. 6C, 6F, and FIGS. 11, 12). These disparate results suggest that plasmid DNA-containing PEM multilayer films remained intact upon microneedle penetration and subsequently release DNA over a period of 24 hours, while PLGA NP-containing PEM multilayer films are likely deposited in the skin concomitantly with microneedle insertion. Without being bound to any theory, it is believed that pDNA undergoes some degree of interpenetration during incorporation in PEMs, consistent with other polyion species. This would lead to molecular entanglements that would not be present in the nanoparticle multilayer films and could account for the relative ease of removal of these films once inserted into the skin. Thus both PEM multilayer architecture and the nature of the encapsulated components are parameters controlling the delivery properties of PEM-coated microneedles. Notably, arrays coated first with (poly-1/pLUC) followed by 4 bilayers of spray-deposited (poly-1/PLGA NPs) co-delivered DNA and PLGA NPs to the skin of live mice after a 24-hr application (FIGS. 6G and 13).

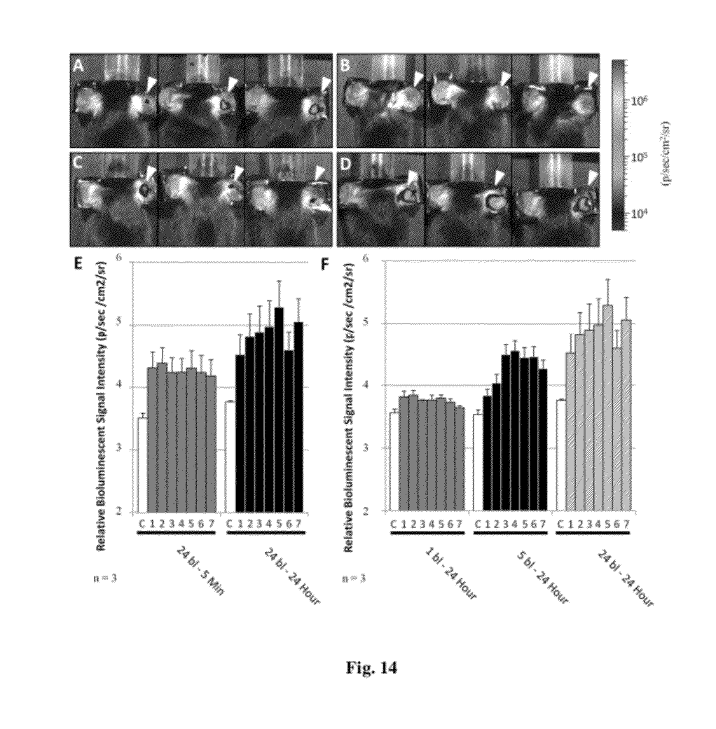

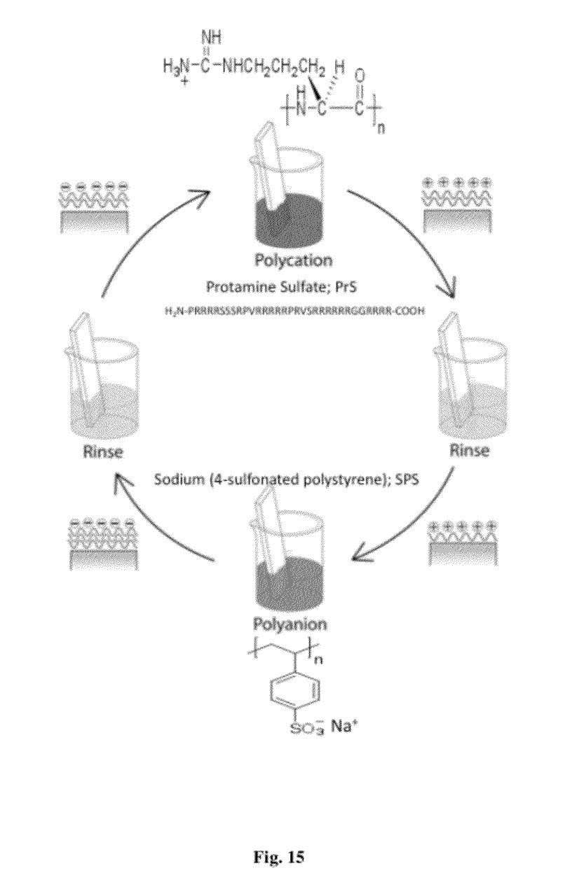

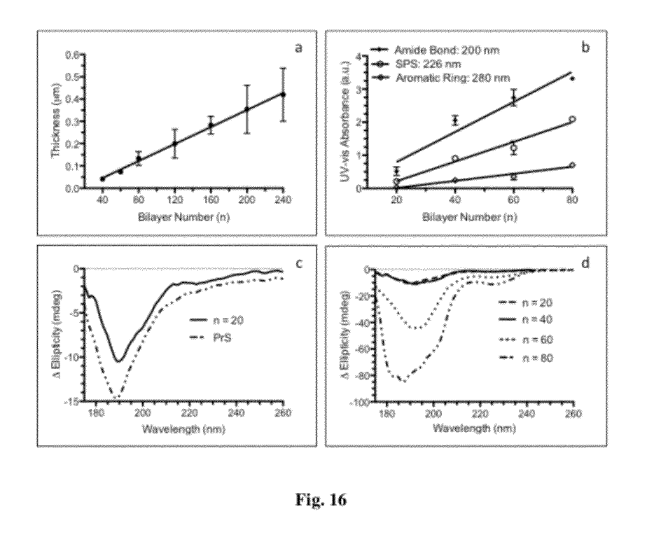

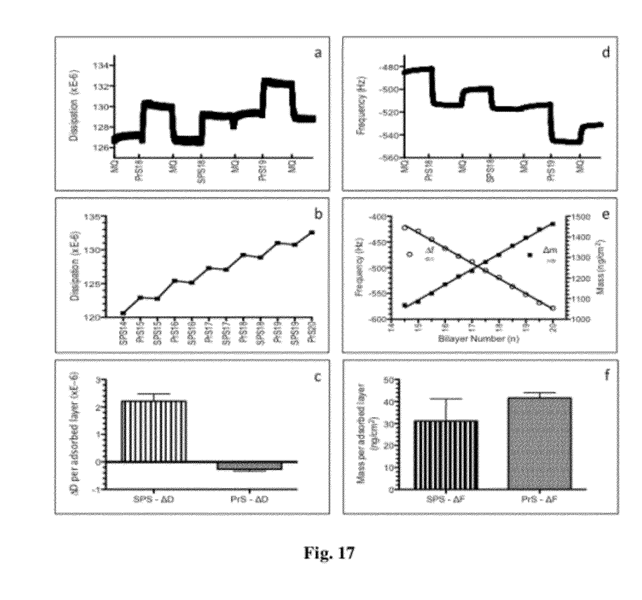

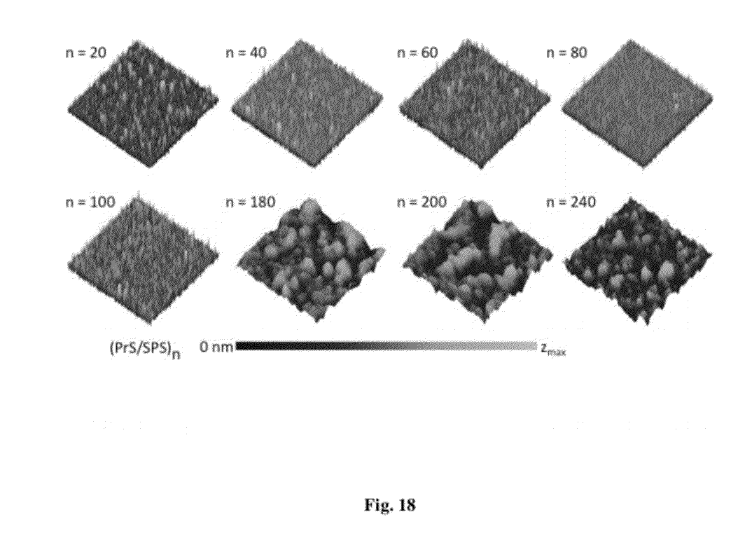

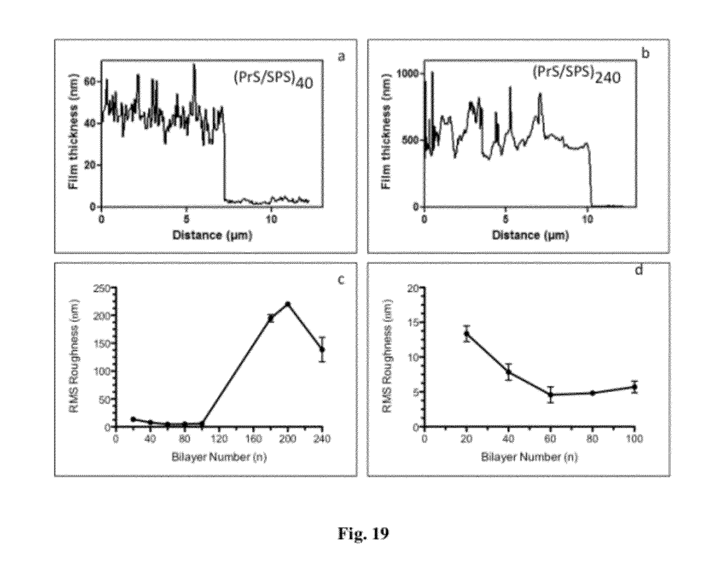

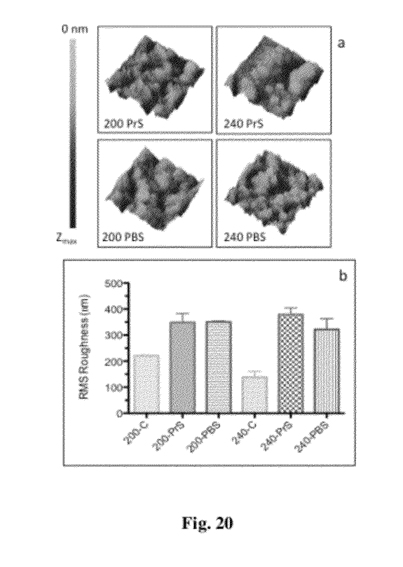

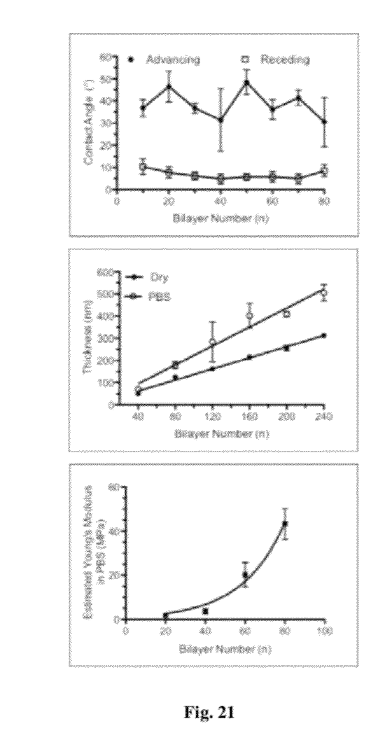

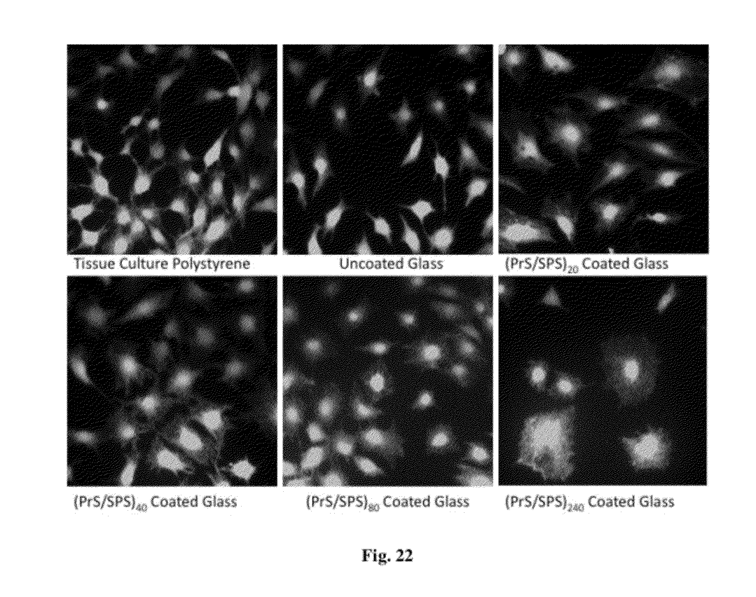

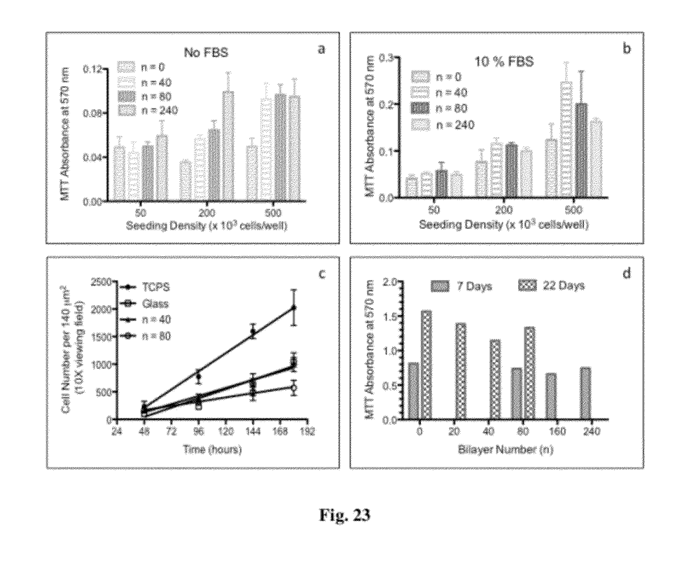

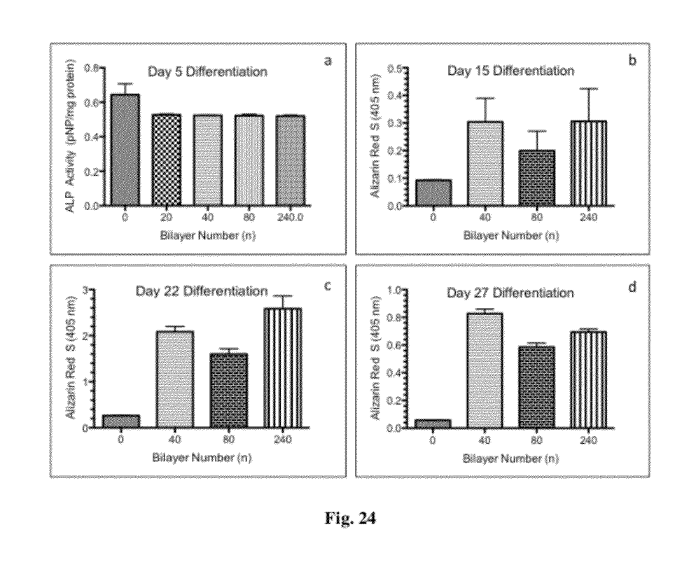

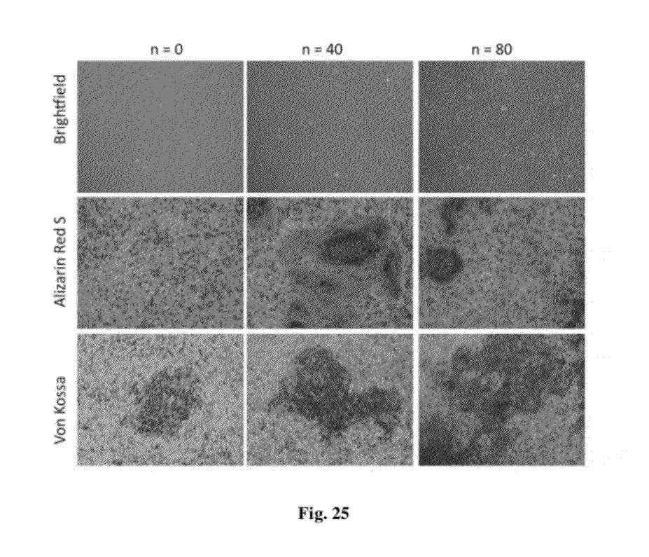

Although murine and human skin exhibit a number of structural differences, preclinical mouse studies of transcutaneous vaccine delivery have been remarkably predictive of clinical trial results. In addition, the mouse model permits a detailed functional analysis of biological responses to delivered pDNA or NPs. In order to further evaluate the potential of PEM-coated microneedle arrays for transcutaneous DNA delivery, we assessed the ability of (poly-1/pLUC)-coated PLGA microneedles to transfect cells in vivo. PEM-coated microneedles were applied to the dorsal ear skin of C57BL/6 mice, and in vivo transfection was quantified over time using whole animal bioluminescence imaging to detect luciferase expression. Mice were treated by application of a 24 bilayer (poly-1/pLUC)-coated microneedle array to ear skin for 5 minutes (FIG. 14), or a 1-(FIG. 14B), 5-(FIG. 14C) or 24-bilayer (FIG. 14D) array for 24 hrs. Bioluminescence was then monitored in vivo for 7 days. Successful in vivo transfection and expression of firefly luciferase in the ear skin was detected for both 5 minute and 24 hr application times, despite the low level of pDNA detected in skin for the former (FIG. 14E). In both cases, luciferase expression was detected for over a week, though pLUC-coated microneedles applied for 24 hours resulted in an increase in the intensity of luciferase expression, as expected from the CLSM results described above. Additionally, the iterative nature of LbL film construction is amenable to robust dosage control. Application of microneedle arrays coated with 1, 5, or 24 bilayers of (poly-1/pLUC) for 24 hours gave luciferase expression levels spanning an order of magnitude (FIG. 14F).

In conclusion, as a first step towards the design of a general materials platform for transcutaneous DNA and therapeutic NP delivery, we have demonstrated for the first time the application of LbL self-assembly for the deposition of functional coatings on microneedle arrays. We have shown the versatility of this approach, engineering PEM films containing pDNA and/or degradable polymer NPs, and demonstrating their utility for delivery into the viable epidermis through microneedle application. Finally, we have shown for the first time to our knowledge, successful in vivo transfection via DNA released from microneedle-supported PEM films. These findings suggest the utility of these materials for DNA vaccine delivery and gene therapy, as well as the co-delivery of therapeutic-loaded degradable polymer NPs for sustained and controlled release of encapsulated materials in vivo.
[1] E. L. Giudice, J. D. Campbell, Adv. Drug Delivery Rev. 2006, 58, 68.
[2] U. Donatus, G. Bruce, T. Robert, Bull World Health Organ 2002, 80, 859.
[3] E. R. A. Pruss-Ustun, Y. Hutin, in WHO Environmental Burden of Disease Series, World Health Organization, 2003.
[4] F.-S. Quan, Y.-C. Kim, D.-G. Yoo, R. W. Compans, M. R. Prausnitz, S.-M. Kang, PLoS One 2009, 4, e7152.
[5] Y.-C. Kim, F.-S. Quan, D.-G. Yoo, W. Compans Richard, S.-M. Kang, R. Prausnitz Mark, J. Infect Dis 2010, 201, 190.
[6] G. M. Glenn, R. T. Kenney, L. R. Ellingsworth, S. A. Frech, S. A. Hammond, J. P. Zoeteweij, Expert Rev. Vaccines 2003, 2, 253.
[7] M. R. Prausnitz, R. Langer, Nat. Biotechnol. 2008, 26, 1261.
[8] H. S. Gill, M. R. Prausnitz, J. Controlled Release 2007, 117, 227.
[9] M. R. Prausnitz, Adv. Drug Delivery Rev. 2004, 56, 581.
[10] B.-S. Kim, S. W. Park, P. T. Hammond, ACS Nano 2008, 2, 386.
[11] D. M. Lynn, Adv. Mater. 2007, 19, 4118.
[12] X. Su, B.-S. Kim, S. R. Kim, P. T. Hammond, D. J. Irvine, ACS Nano 2009, 3, 3719.
[13] N. Jessel, M. Oulad-Abdelghani, F. Meyer, P. Lavalle, Y. Haikel, P. Schaaf, J. C. Voegel, Proc. Natl. Acad. Sci. U. S. A. 2006, 103, 8618.
[14] T. Boudou, T. Crouzier, K. Ren, G. Blin, C. Picart, Adv. Mater. 2010, 22, 441.
[15] F. Cavalieri, A. Postma, L. Lee, F. Caruso, ACS Nano 2009, 3, 234.
[16] M. Dimitrova, C. Affolter, F. Meyer, I. Nguyen, D. G. Richard, C. Schuster, R. Bartenschlager, J.-C. Voegel, J. Ogier, T. F. Baumert, Proc. Natl. Acad. Sci. U. S. A. 2008, 105, 16320.
[17] J. Zhang, L. S. Chua, D. M. Lynn, Langmuir 2004, 20, 8015.
[18] C. M. Jewell, J. Zhang, N. J. Fredin, D. M. Lynn, J. Controlled Release 2005, 106, 214.
[19] M. Pearton, C. Allender, K. Brain, A. Anstey, C. Gateley, N. Wilke, A. Morrissey, J. Birchall, Pharm. Res. 2008, 25, 407.
[20] J. A. Mikszta, J. B. Alarcon, J. M. Brittingham, D. E. Sutter, R. J. Pettis, N. G. Harvey, Nat. Med. 2002, 8, 415.
[21] H. S. Gill, J. Soederholm, M. R. Prausnitz, M. Saellberg, Gene Ther. 2010, No pp yet given.
[22] J.-H. Park, M. G. Allen, M. R. Prausnitz, J. Controlled Release 2005, 104, 51.
[23] M. I. Haq, E. Smith, D. N. John, M. Kalavala, C. Edwards, A. Anstey, A. Morrissey, J. C. Birchall, Biomed Microdevices 2009, 11,35.
[24] D. M. Lynn, R. Langer, J. Am. Chem. Soc. 2000, 122, 10761.
[25] J. R. Greenland, H. Liu, D. Berry, D. G. Anderson, W.-K. Kim, D. J. Irvine, R. Langer, N. L. Letvin, Mol. Ther. 2005, 12, 164.
[26] A. Akinc, D. G. Anderson, D. M. Lynn, R. Langer, Bioconjugate Chem. 2003, 14, 979.
[27] K. C. Wood, J. Q. Boedicker, D. M. Lynn, P. T. Hammond, Langmuir 2005, 21, 1603.
[28] B.-S. Kim, R. C. Smith, Z. Poon, P. T. Hammond, Langmuir 2009, 25, 14086.
[29] C. M. Jewell, D. M. Lynn, Adv. Drug Delivery Rev. 2008, 60, 979.
[31] J. F. Liang, V. C. Yang, Y. Vaynshteyn, Biochem. Biophys. Res. Commun. 2005, 336, 653.
[32] A. Bershteyn, J. Chaparro, R. Yau, M. Kim, E. Reinherz, L. Ferreira-Moita, D. J. Irvine, Soft Matter 2008, 4, 1787.
[33] K. C. Krogman, J. L. Lowery, N. S. Zacharia, G. C. Rutledge, P. T. Hammond, Nat. Mater. 2009, 8, 512.
[34] M. Boes, J. Cerny, R. Massol, M. Op den Brouw, T. Kirchhausen, J. Chen, L. Ploegh Hidde, Nature 2002, 418, 983.
[35] M. D. Abramoff, P. J. Magelhaes, S. J. Ram, Biophotonics International 2004, 11, 36.

### Example 2

Materials: Protamine sulfate (PrS; MW=4,500 Da), poly(sodium 4-styrenesulfonate) (SPS; MW=70,000 Da), and tetrazolium [3-(4,5-dimethylthiazol-2-yl)-2,5-diphenyltetrazolium bromide] (MTT) were purchased from Sigma-Aldrich (St. Louis, Mo.). Alpha-minimum essential medium (α-MEM), fetal bovine serum (FBS), antibiotic-antimycotic solution, trypsin-EDTA, Hoechst 33242, and the Live/Dead® Viability/Cytotoxicity Kit for mammalian cells (L3342) were obtained from Invitrogen (Carlsbad, Calif.). Sodium acetate solution was purchased from Lonza (Portland, Me.). Test grade n-type silicon wafers, quartz slides, and glass slides were obtained from Silicon Quest (Santa Clara, Calif.), Chemglass (Vineland, N.J.), and VWR International (West Chester, Pa.), respectively. Deionized water (18.2 M S2 , Milli-Q Ultrapure Water System, Millipore) was utilized in all experiments.

Fabrication of PEM: LbL film assembly was performed using an automated slide stainer as previously described. In brief, the substrate (silicon, quartz, or glass) was cleaned sequentially in methanol, ethanol, methanol and water, dried with filtered nitrogen, plasma etched for 5 minutes at high RF setting, and then immediately immersed in the cationic PS solution (2 mg/ml in 0.1 M sodium acetate buffer, pH 5.0) for at least 15 minutes prior to commencing the automated dipping protocol. The LbL protocol was designed to produce a bilayer PEM architecture through the alternating immersion of the substrate in the polycation (PrS) solution, two water rinses, the polyanion solution (SPS; 20 mM with respect to the polymer repeat unit), and two additional rinses of water This dipping protocol was repeated n times to produce the final PEM designated (PrS/SPS)_{n,} where n represents the number of bilayers deposited (FIG. 15). Upon fabrication the PEMs were dried with filtered nitrogen and stored in sealed vials at room temperature.

Dry Characterization of (PrS/SPS)ₙ PEMs: Atomic force microscopy (AFM) imaging of the surface morphology of dry (PrS/SPS). PEMs at n=20, 40, 60, 80, 100, 180, 200, and 240 bilayers was conducted. A MultiMode 8 scanning probe microscope with a Nanoscope V controller from Veeco Metrology (Santa Barbara, Calif.) operated in Peak Force Tapping mode was utilized for all measurements. ScanAsyst software (Veeco) was used to map film height. Film morphology was tracked using silicon probes over a 10 μm×10 μm area. Film root mean squared (RMS) roughness values were determined using NanoScope Analysis 1.10 software (Veeco). The thickness of dry (PrS/SPS)ₙ PEMs were determined using a Tencor P16 profilometer as previously reported, with stylus force of 2 mg and scan length of 1 mm.

UV-Vis spectroscopy was used to determine the accumulation of PrS (amide bond at 200 nm and aromatic ring at 280 nm) and SPS (226 nm) during the PEM growth process. UV-Vis spectra were obtained from PEMs fabricated on quartz substrates using a VarianCary 6000i UV-Vis-NIR spectrophotometer. Circular dichroism (CD) was performed on dry PEMs fabricated on quartz substrates in order to determine whether the secondary structure of PrS was altered during film incorporation using an Aviv Biomedical 202 Circular Dichroism Spectrophotometer. Scan ranges spanned 300 nm to 170 nm. Difference spectra were obtained by subtracting baseline corrected (uncoated quartz slide) spectra of dry (PrS/SPS)ₙ thin films oriented 90° relative to each other.

Liquid Phase Characterization of PEMs: Quartz Crystal Microbalance with Dissipation (QCM-D) was used to monitor the in situ deposition of the polyelectrolytes (PrS and SPS) on SiO₂ coated QCM-D quartz cystals. The resonance frequencies (overtones) of the crystals were monitored on a D300 QCM-D (Q-Sense, Inc.). The absolute resonance frequency (f) and the absolute dissipation (D) of at least four overtones were measured during the 10 minute flow period for the polycation solution, the Milli-Q water rinse, the polyanion solution, and the final Milli-Q water rinse of each deposited bilayer. All measurements were acquired at 25° C. Data was analyzed with aid of Q-Tools software (Q-Sense, Inc.).

Dynamic air-water contact angle measurements of (PrS/SPS)ₙ PEMs were obtained using the sessile drop method on a Rame-Hart Contact Angle Goniometer. Advancing and receeding contact angles were measured after depositing 4 μl of MilliQ water to the surface. Spectroscopic ellipsometry (Woollam WVASE Spectroscopic Ellipsometer) was used to study the in situ swelling response of the PEMs. PEM thicknesses were measured in the dry state and after hydration in phosphate buffered saline, pH 7.2 without calcium and magnesium. All thickness measurements were obtained at room temperature with the light source at a 70° angle of incidence. The PEM thickness was determined by fitting the spectra with a Cauchy dispersion model.

The estimated Young's moduli of hydrated 20, 40, 60, and 80 bilayer PEMs were obtained in a fluid AFM cell on the Veeco AFM described earlier. The PEMs were hydrated in approximately 100 μL of 0.01 M PBS, pH 7.2 for all measurements. (PrS/SPS)ₙ PEM moduli were tracked over a 10 μm×10 μm area using silicon nitride probes in solution. The PeakForce Quantitative Nano-mechanical Property Mapping (PeakForce QNM) capabilities from Veeco were used to estimate the Young's moduli. The NanoScope 8.1 software (Veeco) utilizes the DMT model to estimate the Young's moduli. Average estimated Young's moduli were obtained using NanoScope Analysis 1.10 software (Veeco). Three separate films samples were used for all measurements with 3 to 5 images taken per sample.

MC3T3-E1 Cell Culture on (PrS/SPS)ₙ PEMs: A mouse pre-osteoblast cell line (MC3T3-E1 Subclone 4; American Type Culture Collection; ATCC; CRL-2594) was used for all PEM-cell interaction studies. MC3T3-E1 cells were cultured in TCPS in a growth medium consisting of α-MEM, 10% FBS, and 1% of antibiotic-antimycotic solutionand maintained in a humidified incubator (37° C.; 5% CO₂ in air). Culture medium was replenished every 2-3 days. MC3T3-E1 cells were sub-cultured when near 100% confluence with the use of 0.05% trypsin-EDTA solution. All MC3T3-E1 cells used in these studies were less than passage number 12.

Cell Adhesion Assays: MC3T3-E1 cells were seeded at a density of 50,000, 200,000, and 500,000 cells/well in 6-well TCPS plates containing 24 mm×25 mm glass substrates which were either non-coated controls orcoated with 40, 80, or 240 bilayers of (PrS/SPS)ₙ. The test of cell adhesion to the substrates was performed in both α-MEM without FBS and with 10% FBS. Cells were cultured at 37° C. and 5% CO₂ in humidified air for two hours prior to determination of cellular metabolic activity by the use of the MTT assay and direct measurement of cell numbers by the Live/Dead® Viability/Cytotoxicity Kit supplemented with Hoechst 33342 as previously described.

Cell Proliferation Assays: The ability of MC3T3-E1 cells to proliferate on (PrS/SPS)ₙ PEMs was evaluated by the MTT assay and Live/Dead® Viability/Cytotoxicity assay described above. MC3T3-E1 cells in growth media were seeded (50,000 cells/well) into 6-well TCPS plates containing PEMs and allowed to proliferate until becoming fully confluent. Samples were sequentially evaluated using the MTT assay and the Live/Dead® assay at time points from 48 hours to more than one-week after being seeded onto the PEMs and control surfaces (uncoated glass and TCPS).

Cell Differentiation Assays: Experiments were also performed in order to evaluate the ability of MC3T3-E1 cells to differentiate into mature osteoblasts while adherent to the surface of (PrS/SPS) PEMs. Cells were initially seeded (500,000 cells/well) onto uncoated and film-coated glass substrates 48 hours prior to the induction of MC3T3-E1 differentiation by replacing the growth media with differentiating media (growth media described above supplemented with 50 μg/ml L-ascorbic acid and 10 mM β-glycerophosphate). The presence of alkaline phosphatase (an early marker of osteogenic differentiation) was evaluated quantitatively (enzyme activity) five days after addition of the differentiation media. The accumulation of calcium within the differentiating MC3T3-E1 cell culture was likewise assessed both qualitatively and quantitatively by Alizarin red S (ARS; Sigma). The maturation of the deposited calcium was demonstrated by the staining of the hydroxyapaptite crystals (containing calcium and phosphorous) by the silver nitrate-based Von Kossa staining protocol.

Statistical Analysis: All data analysis was performed in GraphPad Prism 5 Software (San Diego, Calif.). Data are reported as mean±standard deviation of a minimum of at least 3 samples. Statistical significance (P<0.05) was determined by GraphPad Prism 5 software using either Student's two-tailed t-tests or one-way ANOVA using nonparametric Kruskal-Wallis test and Dunns' post-hoc analysis.

Film Thickness and Growth Behavior of (PrS/SPS)ₙ PEMs: Bilayer architecture PEMs were constructed using the cationic PrS and anionic sodium (4-polystyrene sulfonate) (SPS), and the nature of film growth and their surfaces were characterized. Cleaned silicon substrates were functionalized with these (PrS/SPS)ₙ films over a wide range of bilayer numbers (n). A schematic of the components used in film assembly and the film architecture are shown in FIG. 15. The thickness of the (PrS/SPS)ₙ PEMs increased linearly as seen in FIG. 16a, with a relatively small incremental increase per bilayer pair (1.91±0.06 nm per bilayer; R²=0.77). The accumulation of the individual components within the thin film was monitored by UV-Vis spectroscopy of films deposited on quartz substrates (FIG. 16b). This allowed serial monitoring of the increase in PrS (amide bond and aromatic ring absorption maxima at 200 nm and 280 nm, respectively) and SPS (absorption maximum at 226 nm). There were progressive increases in absorption intensity at the 200 nm, 226 nm, and 280 nm wavelengths with increasing bilayer number as shown in FIG. 16b. However, the amide bond wavelength (200 nm; 0.043±0.003 a.u./nm) was an exceedingly more sensitive measure of the accumulation of PrS than the aromatic ring (280 nm; 0.005±0.001 a.u./nm) wavelength because protamine contains primarily aliphatic amino acids and only one aromatic amino acid. The rates of increase in the film components were determined from the slopes of linear regression lines in FIG. 16b and showed that PrS (0.043±0.003 a.u./bilayer) increased at the same order of magnitude as SPS (0.019±0.002 a.u./bilayer), suggestive of similar mass contribution of both components to the increase in thickness of the (PrS/SPS)ₙ PEMs.

Circular dichroism (CD) analysis was used to determine whether the electrostatic deposition of the PrS polypeptide within the structure of the (PrS/SPS)ₙ PEMs altered its secondary structure. Deposition of single layers of PrS (FIG. 16c) confirmed previously published results showing that the de novo secondary structure of PrS is that of a random coil. CD spectra of dry (PrS/SPS)ₙ PEM functionalized quartz surfaces showed that the characteristic random coil spectra of PrS in solution was obtained for PrS in the solid state (FIG. 16c-d). The amplitude of the spectra continually increased with an increasing number of deposited layers within the PEMs. Although an additional optically active species was seen in the (PrS/SPS)₈₀ PEM, the spectra still maintained the characteristic random coil secondary structure. This result differs from observations of polypeptide multilayer films fabricated from short peptides, which possessed a random coil conformation in solution and a sheet conformation in the PEM. Spectroscopic analyses of (PrS/SPS)ₙ PEMs confirmed that protamine maintained its native secondary structure when complexed with SPS in the dry, solid thin films. The (PrS/SPS)ₙ film linear growth produced relatively thin coatings over many cycles of polymer deposition, consistent with the complexation of two fully ionized polyelectrolytes with high charge density.

Quartz crystal microbalance with dissipation monitoring (QCM-D) was used for real-time monitoring of (PrS/SPS)ₙ deposition on silicon dioxide coated crystals, the results from which are shown in Supplementary Data (FIG. 17.) QCM-D was used to fabricate (PrS/SPS)₂₀ PEMs with simultaneous monitoring of the frequency (f), which is related to the true mass of polymer deposited on the crystal (m), and the energy dissipation (D) which is related to the viscoelasticity of the deposited mass. SPS was the primary contributor to the progressive increase in dissipation (ΔD=2.21±0.33×10⁻⁶ per SPS layer) during PEM growth; whereas, the adsorption of PrS resulted in a consistent reduction in the dissipation (ΔD=−0.26±0.06×10⁻⁶ per PS layer) during PEM growth (FIG. S1c). Hence, the PEM became somewhat more rigid when PrS was the outer layer, but softer and more dissipative when SPS was the outer layer. The Sauerbrey relation can be assumed to be a good approximation for the (PrS/SPS)ₙ PEMs due to the relatively small changes in ΔD. The post-rinse frequency decreased linearly at a rate of 29.6±0.4 Hz/bilayer (R²=0.99), analogous to an estimated mass deposited of 74.8±1.1 ng/cm²-bilayer computed from Sauerbrey's relation (R²=0.99). Both polymer deposition cycles resulted in near equal change in the frequency (Δf=−12.3±4.0 and −16.4±0.9 Hz for SPS and PrS, respectively), suggesting that each polymer contributes similar polymer mass to the linearly growing (PrS/SPS), PEMs (31.1±10.1 and 41.6±2.4 ng/cm² for each adsorbed layer of SPS and PrS, respectively. These QCM-D results are consistent with the linear increases in PrS and SPS absorbance monitored via UV-Vis and clearly suggest that PrS and SPS, while contributing a similar mass during film growth, result in markedly different alterations in the viscoelastic properties of the thin film with each deposition step.

Thin Film Surface Characterization of (PrS/SPS)ₙ PEMs The films produced are relatively smooth at lower n, but become much rougher at higher n as shown in FIG. 18. A uniform surface morphology consisting of minute surface elevations with maximum heights (zₘₐₓ) in the range of 30 to 110 nm are seen for all (PrS/SPS)ₙ with n≦100 bilayers. A much broader distribution of islands and larger surface features with zₘₐₓ between 1000 to 1600 nm were seen for n 100 bilayers. AFM images of scratched (PrS/SPS)ₙ PEMs fabricated on silicon substrates demonstrated complete surface coverage in all cases (FIG. 19a-b); thus, although the thicker films are rough, they do not show signs of degradation or deconstruction during the assembly process. Surface roughness measurements of the dry (PrS/SPS)ₙ films were obtained by AFM and are shown in FIG. 19(c-d, where d contains a close-up of the film roughness in the first 100 layers). The RMS roughness (R_{q}) progressively decreased as the thickness increased from 20 to 60 bilayers (13.4±1.1 nm to 4.6±1.1 nm), remained relatively constant at intermediate film thicknesses (4.6±1.1 nm, 4.8±0.4 nm, and 5.7±0.8 nm at 60, 80, and 100 bilayers, respectively), and drastically increased at the higher bilayer numbers (194.0±6.5 nm, 220.0±2.8 nm, and 138.5±21.9 nm at 180, 200, and 240 bilayers, respectively). This irregular pattern of change in the roughness of (PrS/SPS)ₙ PEMs is markedly different from the constant roughness or linear increase in roughness with increase in thickness typically reported for PEMs. In order to determine whether PrS interlayer diffusion or exchange plays a role in the change of roughness of the films with number of layers, we examined the RMS roughness of 200 and 240 bilayer PrS/SPS PEMs, and the same PEMs after equilibrating in a 10 mM PrS solution and phosphate buffered saline (PBS) for approximately 24 hours. (Supplementary Data, FIG. 20). PrS equilibrated PEMs demonstrated significant increases in surface roughness compared to the native PEMs; similar results were also observed when films were conditioned in the presence of PBS. The fact that these films become rougher when they are simply “annealed” in protamine or buffered salt solutions suggests that over extended periods, the PrS may be sufficiently mobile within the multilayer to allow significant rearrangements of the film during its construction at thicker layers. There may be a critical film thickness beyond which this film rearrangement is favored based on the balance between surface interactions and the interactions of the film components within the film matrix. A mechanism involving significant amounts of interdiffusion of PrS within the PEM would typically suggest an exponential increase in thickness with increasing bilayer number; however, this behavior was not observed here.

Dynamic (advancing and receding) air-water contact angle measurements were performed in order to assess the wettability of (PrS/SPS)ₙ PEMs as shown in FIG. 21. The advancing contact angles of 10 to 80 bilayer films)(30-50° were significantly higher than the associated receding contact angles) (5-10°). Dynamic air-water contact angles were not measurable for 100 to 240 bilayer PEMs due to the extreme hydrophilicity of these surface coatings. Hence, the contact angle of (PrS/SPS)ₙ films progressively decreased with increasing number of bilayers, rendering them extremely hydrophilic. In situ liquid-phase spectroscopic ellipsometry was used to investigate the post-fabrication swelling that takes place upon hydration of a dry film. Thicknesses of PEMs submerged in PBS were determined after 5 minutes of hydration (FIG. 21b). The dry-state thicknesses of the PEMs as measured by ellipsometry prior to hydration were consistent with the thicknesses measured by profilometry, and demonstrated the same linear increase with increasing number of bilayers (1.3±0.04 nm/bilayer, R²=0.98). Hydrated thickness of these PEMs showed linear thickness increase with increasing bilayer number, as well (2.1±0.2 nm/bilayer, R²=0.91). The hydrated PEMs were approximately 60.7±18.3% thicker than the corresponding dry films. The thicker (≧300 nm) PEMs were generally swollen to a greater extent (60-80%) compared to the thinner (≦200 nm) PEMs (40-50%). The mechanical properties of the swollen (PrS/SPS)ₙ PEMs were evaluated via liquid-phase AFM. The estimated Young's moduli of the PEMs increased exponentially (R²=0.98) from 20 to 80 bilayers shown in FIG. 21c. In particular, the Young's modulus increased significantly from 1.8±0.3 MPa at 20 bilayers to 43.3±6.9 MPa at 80 bilayers. For comparison, the estimated Young's modulus for the dry 80 bilayer film was 4,850±90 MPa; hence, the hydration of the film results in about a 100-fold decrease in the stiffness of the thin film due to the uptake of water in the LbL ionically crosslinked matrix. The estimated Young's modulus of these hydrated (PrS/SPS)ₙ PEMs was significantly higher than the 3-400 kPa reported for chemically cross-linked poly(L-lysine)/hyaluronan films, but similar to the 6-100 MPa range reported for hydrated, synthetic weak polyelectrolyte PEMs studied by VanVliet and Rubner. Hydrated (PrS/SPS)ₙ PEM stiffness increased exponentially with bilayer number to magnitudes that usually require post-fabrication cross-linking of the PEM through chemical exposure or heat treatment. The increased surface roughness and stiffness with growth of the (PrS/SPS)ₙ PEMs are expected to be compatible with enhancing cell-PEM interactions. The moderately hydrophilic nature of the (PrS/SPS)ₙ PEMs should also prove useful in promoting cell-PEM interactions.

A rigid support is essential for the proper interaction of cells with their underlying scaffold. Our QCM-D analysis showed that incorporation of SPS accounted for the majority of the reduction in the stiffness during assembly, while the addition of PrS markedly increased the PEM stiffness. Conformational changes in the adsorbed polymer layers, as evidenced by changes in dissipation, suggest that SPS may adsorb in a partially shielded, loopy conformation on the surface while the adsorbed PrS appears to be more closely bound to the (PrS/SPS)ₙ PEM surface, potentially forming compact ionic complexes with SPS.⁽⁵⁰⁾ The stiffening of the PEM upon the incorporation of PrS is consistent with PrS behaving as a short, stiff rod-like polyelectrolyte due to the high level of intra-polymer repulsion and the helical backbone in the presence of fully ionized arginine side groups in the acidic (pH 5.0) LbL assembly environment. Hence, the QCM-D results strongly suggest that the unique mechanical properties of the native, non-cross-linked (PrS/SPS)ₙ PEMs, as evidenced by the exponential increase in the Young's modulus in our AFM studies, may arise from the intrinsic rigidity of the short polypeptide PrS as governed by electrostatic repulsive forces.

Osteoconductive Properties of (PrS/SPS)ₙ PEMs

Pre-osteoblast adhesion: (PrS/SPS)ₙ PEMs were investigated for their ability to support the adhesion of cells in culture as a function of number of bilayers or film thickness. MC3T3-E1 cells maintained their normal polygonal morphology with multiple cellular projections both on control substrates (tissue culture polystyrene(TCPS) and uncoated glass) and on (PrS/SPS)ₙ functionalized surfaces (FIG. 22). Sub-confluent monolayers of MC3T3-E1 pre-osteobalst cells assumed an elongated, spindle-like morphology with low cytoplasm area when adherent to TCPS and uncoated glass substrates. In contrast, these cells assumed a cuboidal morphology with marked increase in cytoplasm area and numerous surface projections when adherent to PrS/SPS coated substrates. The lower (20 and 40) bilayer PrS/SPS coated surfaces show MC3T3-E1 cells with numerous cytoplasm projections asymmetrically distributed around the cell nucleus. The higher (80 and 240) bilayer PrS/SPS coated surfaces showed a decreased number and shorter cytoplasm projections, but the cells maintained a high cytoplasm area.

The adhesion of MC3T3-E1 cells to each substrate (either uncoated glass or PEM coated glass) was quantified by cellular metabolic activity (MTT assay) normalized to total culture area as shown in FIG. 23a-b. Serum-free cultures of MC3T3-E1 cells on (PrS/SPS)ₙ PEM functionalized surfaces demonstrated an identical level of cell adhesion as serum-free cultures on uncoated glass surfaces at the lowest cell seeding density (5,000 cells/cm²). In marked contrast, (PrS/SPS)ₙ PEM functionalized surfaces possessed significantly higher serum-free cell adhesion than uncoated glass at the higher cell seeding densities (20,000 and 50,000 cells/cm²). There was generally no statistical difference between MC3T3-E1 adhesion in serum-containing medium on uncoated glass and (PrS/SPS)ₙ PEM functionalized surfaces, except at the highest seeding density (FIG. 23b). Cells generally adhere poorly to non-cross-linked PEMs in direct correlation to their native mechanical properties. The three-fold increase in the stiffness of (PrS/SPS)ₙ PEMs compared to conventional PEMs appears to be primarily responsible for the normalization of cell adhesion to that of cultures on uncoated glass and/or TCPS. Others have previously reported enhanced MC3T3-E1 adhesion to amine terminated silicon oxide substrates with nanometer-scale surface roughness. Enhanced osteoblast adhesion and focal adhesion formation have been demonstrated on nanometer-scale structures on implant surfaces. There was generally no statistical difference in cell adhesion between 40, 80, and 240 bilayer PEMs at all seeding densities, indicating negligible effect of large changes in nanometer-scale roughness on MC3T3-E1 adhesion.

Cell adhesion to all surfaces was best at the highest (50,000 cells/cm²) compared to the lower (5,000 and 20,000 cells/cm²) seeding densities. PEMs generally supported low levels of cell adhesion at low cell seeding density where cell-matrix interactions (integrin binding and focal adhesion formation) are expected to be the major contributor to cell adhesion, but higher levels of cell adhesion at the high seeding density where cell-cell interactions (cadherins) are expected to be a substantial contributor to cell adhesion. Cell adhesion to all surfaces was much higher in 10% fetal bovine serum cultures than in serum-free cultures as shown in FIG. 23b. Hence, binding of serum proteins to the surfaces greatly facilitated initial cell-surface interaction. It appears that the nature of the proteins and/or the magnitude of the protein binding to the hydrophilic uncoated glass and PEMs differs markedly from that of the hydrophobic TCPS surface.

Pre-osteoblast proliferation: MC3T3-E1 pre-osteoblast cells display two distinct growth phases during in vitro osteogenic differentiation. After MC3T3-E1 cells become attached to an osteoconductive surface, they enter a rapid proliferative growth phase in order to establish critical cell-cell interactions essential for the subsequent post-confluent differentiation growth phase. Encouraged by the ability of native (PrS/SPS)ₙ PEM functionalized surfaces to adequately support the adhesion of MC3T3-E1 cells, PEMs were assessed for their ability to support proliferation of cells seeded at a low density (5,000 cells/cm²). MC3T3-E1 cell proliferation was monitored directly via fluorescence microscopy imaging of individual cells directly attached to the culture surfaces and indirectly via the use of the MTT assay to measure the metabolic activity of the total population of cells growing on the surfaces (FIG. 23c-d, respectively). MC3T3-E1 cells proliferated most rapidly on TCPS (13.7±0.6 cells/140 μm²/hr, R²=0.95). In contrast, MC3T3-E1 cells proliferate rates on uncoated glass and 40-bilayer functionalized surfaces (7.0±0.4 and 6.1±0.3 cells/140 μm²/hr, respectively; R²=0.89 and 0.84, respectively), but at the slowest rate on 80-bilayer films (3.2±0.2 cells/140 μm²/hr, R²=0.68). The inverse relation between nanometer-scale surface roughness and osteoblast proliferation demonstrated here have been previously noted on surfaces with micron-scale and nanometer-scale surface roughness. Lower osteoblast proliferation are generally seen on rough surfaces than on smooth surfaces. The MTT activity was higher at 3 weeks than at 1 week in culture (FIG. 23d), indicating the MC3T3-E1 pre-osteoblasts were able to sustain growth on (PrS/SPS)ₙ PEMs over a wide range of PEM thicknesses (n=20, 40, 80, 160, and 240).

Pre-osteoblast differentiation (PrS/SPS)ₙ PEMs were investigated for their ability to support the differentiation of cells in culture. After initial high seeding density (50,000 cells/cm²) in serum-containing growth media and a 48-hour culture stabilization period, the cells were cultured for a subsequent 4 weeks in osteogenic differentiation media. Osteogenic differentiation of MC3T3-E1 cells was assessed by alkaline phosphatase (ALP) enzyme activity, Alizarin Red S (ARS) staining and quantification, and von Kossa staining. Increased ALP enzyme activity is an early marker of osteogenic differentiation. Anionic ARS efficiently stains the calcium deposits in the newly deposited extracellular matrix (ECM) of differentiated osteoblasts. Mineralization of calcified ECM, due to the incorporation of phosphate ions to form the hydroxapatite bone mineral, is commonly visualized by von Kossa staining. Three critical processes are required for bone formation: the presence of osteogenic stem and/or progenitor cells, ostoinductive growth factors to stimulate the differentiation of these cells along an osteoblastic pathway, and an osteoconductive surface to support cell growth and the deposition of new bone matrix. Five days after the induction of osteogenic differentiation, ALP activity of cells on all (PrS/SPS)ₙ PEMs was significantly lower than cells on the uncoated glass as seen in FIG. 24a. A reduction in ALP enzyme activity was previously reported for osteoblasts cultured for 7 days on hydrophilic substrates with micrometer-scale surface roughness. In contrast, quantification of the ARS staining showed that the amount of ARS (calcium deposits) on all (PrS/SPS)ₙ PEMs were significantly greater than that on control uncoated glass slides at 15, 22, and 27 days after the induction of osteogenic differentiation (FIG. 8b-d). There was no statistical difference among the (PrS/SPS)ₙ PEM ARS staining at 15 days as seen in FIG. 8b. The ARS staining on all (PrS/SPS)ₙ PEM functionalized surfaces were greatest at 22 days (FIG. 8c) and was 5 to 10 times higher than ARS levels on uncoated glass surfaces. The ARS staining on the 80 bilayer functionalized glass surfaces were significantly lower than that of the 40 and 240 bilayer PEMs, in a pattern that parallels the surface roughness of these PEMs. A direct association between increased micro-scale roughness and increased osteoblast differentiation has been previously demonstrated by others.

Focal ARS staining of the extracellular matrix was noted in the MC3T3-E1 monolayers on (PrS/SPS)ₙ PEM functionalized surfaces shown in FIG. 25. The intensity of the ARS staining and the size of the focal deposits increased with increasing film thickness. The von Kossa staining was also enhanced on the (PrS/SPS)ₙ PEM functionalized surfaces, but not with as wide differences in staining intensity with increasing bilayers as seen with the ARS staining Nevertheless, the focal areas of von Kossa staining closely match those depicted in the ARS images. Osteointegration of a metallic implant with host bone depends both on the recruitment of stem cells and the induction of these cells to differentiate into osteoblasts (osteoinduction) and on the ability of the implant surface to support the adhesion, proliferation, and differentiation of the osteoblasts leading to the deposition of a mineralized bone matrix on the implant surface (osteoconduction). The enhanced ARS and von Kossa staining of long-term MC3T3-E1 cultures strongly suggest that (PrS/SPS)ₙ functionalized surfaces possess particular physiochemical characteristics that favor the differentiation of osteoblastic progenitors and/or favor the mineralization of the ECM deposited by mature osteoblasts, thus possessing intrinsic osteoconductive properties. The magnitude of calcium deposition increases in direct relation to the amount of PrS and SPS within the functionalized PEM surface. The anionic SPS within the PEMs could potentially serve as binding sites to sequester free calcium and would likely result in diffuse homogeneous calcium deposition, not the large focal calcium deposits observed. Alternatively, the highly positively charged PrS may serve as a binding site for acidic phospholipids and matrix vesicles, both critical for the nucleation of mineralization. The focal nature of the calcium deposits observed on the (PrS/SPS)ₙ PEM functionalized surfaces is more consistent with a PrS-matrix vesicle sequestration process. These matrix vesicles, produced from the cell membrane of the MC3T3-E1 cells, are enriched with acidic phospholipids and contain a high concentration of calcium needed for the nucleation of bone mineral in the extracellular matrix.

Increased ARS staining of the cell layer directly correlated with increasing surface roughness and hydrophilicity of (PrS/SPS)ₙ PEMs. The combination of high nanometer-scale roughness and hydrophilicity resulted in the greatest enhancement of osteoblast differentiation. Moreover, the magnitude of osteogenic differentiation on hydrophilic surfaces can be modulated by relatively small changes in the nanometer-scale roughness. Material surface chemistry and topography are key regulators of osteoblast differentiation at the cell-implant interface. In particular, the osteoconductivity of hydrophilic high surface energy surfaces have been demonstrated by enhanced osteoblastic differentiation in vitro and by improved osteointegration of titanium implants in animal models, as evidenced by increased removal torque forces and bone-to-implant contact values. Recent reports have demonstrated the modulation of human mesenchymal stem cell (hMSC) fate on nanometer-scale structures, wherein 10 nm structures stimulated osteoblastic differentiation, 30 nm nanotubes promoted enhanced adhesion without associated differentiation, and 70-100 nm nanotubes induced hMSC elongation. It is possible that (PrS/SPS)ₙ PEM functionalized surfaces with their hydrophilic surface chemistry and nanometer-scale surface roughness may likewise modulate stem cell fate.

Researchers are actively involved in developing methods to facilitate the use of PEMs for the delivery of osteoinductive factors (growth factors and vectors harboring transgenes) from the surface of orthopedic implants. In contrast to the use of organic osteoinductive agents, the application of osteoconductive materials to the surface of orthopedic implants has primarily focused on the use of inorganic crystalline (calcium phosphate and hydroxyapatite) or ceramic materials. Few studies have explored the use of organic polymers as osteoconductive coatings for orthopedic implant surfaces. Hence, the osteoconductive nature of (PrS/SPS)ₙ functionalized surfaces provides a novel approach towards augmenting bone growth on the surface of orthopedic implants. We have devised a novel protamine-based PEM system that does not require harsh post-fabrication cross-linking treatments to increase PEM stiffness; these films paradoxically increase in stiffness with increased thickness of the PEM, thus greatly facilitating MC3T3-E1 cell-substrate interactions. These protamine-based PEM functionalized surfaces offer the potential to integrate a myriad of bioactive agents within the PEM nanostructure, to enhance the cellular adhesiveness of implant surfaces, and to modulate the response of cells during their interaction with functionalized surfaces. Protamine-based PEM functionalized surfaces can make an immediate impact in the fields of in vitro osteogenic culture of stem cells and assessing the osteogenic potential of novel factors.
1. Petrie T A, Raynor J E, Reyes C D, Burns K L, Collard D M, Garcia A J. The effect of integrin-specific bioactive coatings on tissue healing and implant osseointegration. Biomaterials. 2008 July; 29(19):2849-57.
2. Roach P, Eglin D, Rohde K, Perry C C. Modern biomaterials: a review—bulk properties and implications of surface modifications. J Mater Sci Mater Med. 2007 July; 18(7):1263-77.
3. de Jonge L T, Leeuwenburgh S C, van den Beucken J J, to Riet J, Daamen W F, Wolke J G, et al. The osteogenic effect of electrosprayed nanoscale collagen/calcium phosphate coatings on titanium. Biomaterials. March; 31(9):2461-9.
4. Ramaswamy Y, Wu C, Dunstan C R, Hewson B, Eindorf T, Anderson G I, et al. Sphene ceramics for orthopedic coating applications: an in vitro and in vivo study. Acta Biomater. 2009 October; 5(8):3192-204.
5. Lavos-Valereto I C, Wolynec S, Deboni M C, Konig B, Jr. In vitro and in vivo biocompatibility testing of Ti-6Al-7Nb alloy with and without plasma-sprayed hydroxyapatite coating. J Biomed Mater Res. 2001; 58(6):727-33.
6. Brama M, Rhodes N, Hunt J, Ricci A, Teghil R, Migliaccio S, et al. Effect of titanium carbide coating on the osseointegration response in vitro and in vivo. Biomaterials. 2007 February; 28(4):595-608.
7. Macdonald M L, Samuel R E, Shah N J, Padera R F, Beben Y M, Hammond P T. Tissue integration of growth factor-eluting layer-by-layer polyelectrolyte multilayer coated implants. Biomaterials. February; 32(5):1446-53.
8. Leguen E, Chassepot A, Decher G, Schaaf P, Voegel J C, Jessel N. Bioactive coatings based on polyelectrolyte multilayer architectures functionalized by embedded proteins, peptides or drugs. Biomol Eng. 2007 February; 24(1):33-41.
9. Cini N, Tulun T, Decher G, Ball V. Step-by-step assembly of self-patterning polyelectrolyte films violating (almost) all rules of layer-by-layer deposition. J Am Chem Soc. June 23; 132(24):8264-5.
10. Ariga K, Hill J P, Ji Q. Layer-by-layer assembly as a versatile bottom-up nanofabrication technique for exploratory research and realistic application. Phys Chem Chem Phys. 2007 May 21; 9(19):2319-40.
11. Schlenoff J B. Retrospective on the future of polyelectrolyte multilayers. Langmuir. 2009 Dec. 15; 25(24):14007-10.
12. Ai H, Jones S A, Lvov Y M. Biomedical applications of electrostatic layer-by-layer nano-assembly of polymers, enzymes, and nanoparticles. Cell Biochem Biophys. 2003; 39(1):23-43.
13. Moskowitz J S, Blaisse M R, Samuel R E, Hsu H P, Harris M B, Martin S D, et al. The effectiveness of the controlled release of gentamicin from polyelectrolyte multilayers in the treatment of Staphylococcus aureus infection in a rabbit bone model. Biomaterials. August; 31(23):6019-30.
14. Macdonald M, Rodriguez N M, Smith R, Hammond P T. Release of a model protein from biodegradable self assembled films for surface delivery applications. J Control Release. 2008 Nov. 12; 131(3):228-34.
15. Mehrotra S, Lynam D, Maloney R, Pawelec K M, Tuszynski M H, Lee I, et al. Time Controlled Protein Release from Layer-by-Layer Assembled Multilayer Functionalized Agarose Hydrogels. Adv Funct Mater. Jan. 22; 20(2):247-58.
16. Wood K C, Boedicker J Q, Lynn D M, Hammond P T. Tunable drug release from hydrolytically degradable layer-by-layer thin films. Langmuir. 2005 Feb. 15; 21(4):1603-9.
17. Mansouri S, Winnik F M, Tabrizian M. Modulating the release kinetics through the control of the permeability of the layer-by-layer assembly: a review. Expert Opin Drug Deliv. 2009 June; 6(6):585-97.
18. Blacklock J, Sievers T K, Handa H, You Y Z, Oupicky D, Mao G, et al. Cross-linked bioreducible layer-by-layer films for increased cell adhesion and transgene expression. J Phys Chem B. April 29; 114(16):5283-91.
19. Alves N M, Picart C, Mano J F. Self assembling and crosslinking of polyelectrolyte multilayer films of chitosan and alginate studied by QCM and IR spectroscopy. Macromol Biosci. 2009 Aug. 11; 9(8):776-85.
20. Boudou T, Crouzier T, Auzely-Velty R, Glinel K, Picart C. Internal composition versus the mechanical properties of polyelectrolyte multilayer films: the influence of chemical cross-linking. Langmuir. 2009 Dec. 15; 25(24):13809-19.
21. Hillberg A L, Holmes C A, Tabrizian M. Effect of genipin cross-linking on the cellular adhesion properties of layer-by-layer assembled polyelectrolyte films. Biomaterials. 2009 September; 30(27):4463-70.
22. Zheng H, Berg M C, Rubner M F, Hammond P T. Controlling cell attachment selectively onto biological polymer-colloid templates using polymer-on-polymer stamping. Langmuir. 2004 Aug. 17; 20(17):7215-22.
23. Berg M C, Yang S Y, Hammond P T, Rubner M F. Controlling mammalian cell interactions on patterned polyelectrolyte multilayer surfaces. Langmuir. 2004 Feb. 17; 20(4):1362-8.
24. Mendelsohn J D, Yang S Y, Hiller J, Hochbaum A I, Rubner M F. Rational design of cytophilic and cytophobic polyelectrolyte multilayer thin films. Biomacromolecules. 2003 January-February; 4(1):96-106.
25. Gemici Z, Shimomura H, Cohen R E, Rubner M F. Hydrothermal treatment of nanoparticle thin films for enhanced mechanical durability. Langmuir. 2008 Mar. 4; 24(5):2168-77.
26. Vazquez C P, Boudou T, Dulong V, Nicolas C, Picart C, Glinel K. Variation of polyelectrolyte film stiffness by photo-cross-linking: a new way to control cell adhesion. Langmuir. 2009 Apr. 9; 25(6):3556-63.
27. Thompson M T, Berg M C, Tobias I S, Lichter J A, Rubner M F, Van Vliet K J. Biochemical functionalization of polymeric cell substrata can alter mechanical compliance. Biomacromolecules. 2006 June; 7(6):1990-5.
28. Carrell D T, Emery B R, Hammoud S. The aetiology of sperm protamine abnormalities and their potential impact on the sperm epigenome. Int J Androl. 2008 December; 31(6):537-45.
29. Brange J, Langkjaer L. Insulin formulation and delivery. Pharm Biotechnol. 1997; 10:343-409.
30. Davis D, Akhtar U, Keaster B, Grozinger K, Washington L, Kelsey S, et al. Challenges and potential for RNA nanoparticles (RNPs). J Biomed Nanotechnol. 2009 February; 5(1):36-44.
31. Moran M C, Pais A A, Ramalho A, Miguel M G, Lindman B. Mixed protein carriers for modulating DNA release. Langmuir. 2009 Sep. 1; 25(17):10263-70.
32. Balabushevich N G, Larionova N I. Protein-loaded microspheres prepared by sequential adsorption of dextran sulphate and protamine on melamine formaldehyde core. J Microencapsul. 2009 November; 26(7):571-9.
33. Shutava T G, Balkundi S S, Vangala P, Steffan J J, Bigelow R L, Cardelli J A, et al. Layer-by-Layer-Coated Gelatin Nanoparticles as a Vehicle for Delivery of Natural Polyphenols. ACS Nano. 2009 Jul. 28; 3(7):1877-85.
34. Shukla A, Fleming K E, Chuang H F, Chau T M, Loose C R, Stephanopoulos G N, et al. Controlling the release of peptide antimicrobial agents from surfaces. Biomaterials. 2010 March; 31(8):2348-57.
35. DeMuth P C, Su X, Samuel R E, Hammond P T, Irvine D J. Nano-layered microneedles for transcutaneous delivery of polymer nanoparticles and plasmid DNA. Adv Mater. Nov 16; 22(43):4851-6.
36. Niemiec W, Zapotoczny S, Szczubialka K, Laschewsky A, Nowakowska M. Nanoheterogeneous multilayer films with perfluorinated domains fabricated using the layer-by-layer method. Langmuir. July 20; 26(14):11915-20.
37. Brewer L, Corzett M, Balhorn R. Condensation of DNA by spermatid basic nuclear proteins. J Biol Chem. 2002 Oct. 11; 277(41):38895-900.
38. Toniolo C, Bonora G M, Marchiori F, Bonin G, Filippi B. Protamines. II. Circular dichroism study of the three main components of clupeine. Biochim Biophys Acta. 1979 Feb. 26; 576(2):429-39.
39. Haynie D T, Zhang L, Zhao W, Rudra J S. Protein-inspired multilayer nanofilms: science, technology and medicine. Nanomedicine. 2006 September; 2(3):150-7.
40. Porcel C, Lavalle P, Ball V, Decher G, Senger B, Voegel J C, et al. From exponential to linear growth in polyelectrolyte multilayers. Langmuir. 2006 Apr. 25; 22(9):4376-83.
41. Dixon M C. Quartz crystal microbalance with dissipation monitoring: enabling real-time characterization of biological materials and their interactions. J Biomol Tech. 2008 July; 19(3):151-8.
42. Johannsmann D, Reviakine I, Rojas E, Gallego M. Effect of sample heterogeneity on the interpretation of QCM(-D) data: comparison of combined quartz crystal microbalance/atomic force microscopy measurements with finite element method modeling. Anal Chem. 2008 Dec. 1; 80(23):8891-9.
43. Feiler A A, Sahlholm A, Sandberg T, Caldwell K D. Adsorption and viscoelastic properties of fractionated mucin (BSM) and bovine serum albumin (BSA) studied with quartz crystal microbalance (QCM-D). J Colloid Interface Sci. 2007 Nov. 15; 315(2):475-81.
44. Porcel C, Lavalle P, Decher G, Senger B, Voegel J C, Schaaf P. Influence of the polyelectrolyte molecular weight on exponentially growing multilayer films in the linear regime. Langmuir. 2007 Feb. 13; 23(4):1898-904.
45. Seo J, Lutkenhaus J L, Kim J, Hammond P T, Char K. Effect of the layer-by-layer (LbL) deposition method on the surface morphology and wetting behavior of hydrophobically modified PEO and PAA LbL films. Langmuir. 2008 Aug. 5; 24(15):7995-8000.
46. Schmidt D J, Cebeci F C, Kalcioglu Z I, Wyman S G, Ortiz C, Van Vliet K J, et al. Electrochemically controlled swelling and mechanical properties of a polymer nanocomposite. ACS Nano. 2009 Aug. 25; 3(8):2207-16.
47. Crouzier T, Picart C. Ion pairing and hydration in polyelectrolyte multilayer films containing polysaccharides. Biomacromolecules. 2009 Feb. 9; 10(2):433-42.
48. Thompson M T, Berg M C, Tobias I S, Rubner M F, Van Vliet K J. Tuning compliance of nanoscale polyelectrolyte multilayers to modulate cell adhesion. Biomaterials. 2005 December; 26(34):6836-45.
49. Richert L, Lavalle P, Vautier D, Senger B, Stoltz J F, Schaaf P, et al. Cell interactions with polyelectrolyte multilayer films. Biomacromolecules. 2002 November-December; 3(6):1170-8.
50. Roach P, Farrar D, Perry C C. Interpretation of protein adsorption: surface-induced conformational changes. J Am Chem Soc. 2005 Jun. 8; 127(22):8168-73.
51. El-Ghannam A R, Ducheyne P, Risbud M, Adams C S, Shapiro I M, Castner D, et al. Model surfaces engineered with nanoscale roughness and RGD tripeptides promote osteoblast activity. J Biomed Mater Res A. 2004 Mar. 15; 68(4):615-27.
52. Pareta R A, Reising A B, Miller T, Storey D, Webster T J. An understanding of enhanced osteoblast adhesion on various nanostructured polymeric and metallic materials prepared by ionic plasma deposition. J Biomed Mater Res A. March 1; 92(3):1190-201.
53. Biggs M J, Richards R G, Gadegaard N, Wilkinson C D, Oreffo R O, Dalby M J. The use of nanoscale topography to modulate the dynamics of adhesion formation in primary osteoblasts and ERK/MAPK signalling in STRO-1+ enriched skeletal stem cells. Biomaterials. 2009 October; 30(28):5094-103.
54. Saha K, Pollock J F, Schaffer D V, Healy K E. Designing synthetic materials to control stem cell phenotype. Curr Opin Chem Biol. 2007 August; 11(4):381-7.
55. Absolom D R, Zingg W, Neumann A W. Protein adsorption to polymer particles: role of surface properties. J Biomed Mater Res. 1987 February; 21(2):161-71.
56. Quarles L D, Yohay D A, Lever L W, Caton R, Wenstrup R J. Distinct proliferative and differentiated stages of murine MC3T3-E1 cells in culture: an in vitro model of osteoblast development. J Bone Miner Res. 1992 June; 7(6):683-92.
57. Keselowsky B G, Wang L, Schwartz Z, Garcia A J, Boyan B D. Integrin alpha(5) controls osteoblastic proliferation and differentiation responses to titanium substrates presenting different roughness characteristics in a roughness independent manner. J Biomed Mater Res A. 2007 Mar. 1; 80(3):700-10.
58. Rausch-fan X, Qu Z, Wieland M, Matejka M, Schedle A. Differentiation and cytokine synthesis of human alveolar osteoblasts compared to osteoblast-like cells (MG63) in response to titanium surfaces. Dent Mater. 2008 January; 24(1):102-10.
59. Bonewald L F, Harris S E, Rosser J, Dallas M R, Dallas S L, Camacho N P, et al. von Kossa staining alone is not sufficient to confirm that mineralization in vitro represents bone formation. Calcif Tissue Int. 2003 May; 72(5):537-47.
60. Schwarz F, Wieland M, Schwartz Z, Zhao G, Rupp F, Geis-Gerstorfer J, et al. Potential of chemically modified hydrophilic surface characteristics to support tissue integration of titanium dental implants. J Biomed Mater Res B Appl Biomater. 2009 February; 88(2):544-57.
61. Albrektsson T, Johansson C. Osteoinduction, osteoconduction and osseointegration. Eur Spine J. 2001 October; 10 Suppl 2:S96-101.
62. Liao H, Andersson A S, Sutherland D, Petronis S, Kasemo B, Thomsen P. Response of rat osteoblast-like cells to microstructured model surfaces in vitro. Biomaterials. 2003 February; 24(4):649-54.
63. Park J W, Jong J H, Lee C S, Hanawa T. Osteoconductivity of hydrophilic microstructured titanium implants with phosphate ion chemistry. Acta Biomater. 2009 July; 5(6):2311-21.
64. Dalby M J, Gadegaard N, Tare R, Andar A, Riehle M O, Herzyk P, et al. The control of human mesenchymal cell differentiation using nanoscale symmetry and disorder. Nat Mater. 2007 December; 6(12):997-1003.
65. Oh S, Brammer K S, Li Y S, Teng D, Engler A J, Chien S, et al. Stem cell fate dictated solely by altered nanotube dimension. Proc Natl Acad Sci U S A. 2009 Feb. 17; 106(7):2130-5.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A structure, comprising:
a substrate;
a multilayer film on the substrate,
wherein the multilayer film comprises a first plurality of first units, each first unit comprising a protamine polypeptide.

2. The structure of claim 1, wherein the multilayer film further comprises a second plurality of second units.

3. The structure of claim 1, wherein the protamine polypeptide is in a salt form.

4. The structure of claim 1, wherein the first plurality of the first units comprises 8, 10, 20, 40, 80 or 240 first units.

5. The structure of claim 2, wherein the second plurality of the second units is between the substrate and the first plurality of the first units.

6. The structure of claim 2, wherein the first plurality of the first units is between the substrate and the second plurality of the second units.

7. The structure of claim 2, wherein at least one of the first plurality of the first units and the second plurality of the second units comprises alternating polycationic and polyanionic layers, and degradation of the multilayer film is characterized by hydrolytic degradation of at least a portion of a member of the polycationic layers, the polyanionic layers, and both.

8. The structure of claim 2, wherein at least portion of the multilayer film comprises a polyelectrolyte.

9. The structure of claim 8, wherein the degradable polyelectrolyte comprises a polymer selected from polyester, polyanhydride, polyorthoester, polyphosphazene, polyphosphoester, and any combination thereof

10. The structure of claim 9, the polyester is selected from a group consisting of poly(β-amino ester)s, poly(L-lactide-co-L-lysine), poly(serine ester), poly(4-hydroxy-L-proline ester), poly[.alpha.-(4-aminobutyl)-L-glycolic acid], and any combination thereof

11. The structure of claim 10, wherein the poly(β-amino ester) is selected from the group consisting of 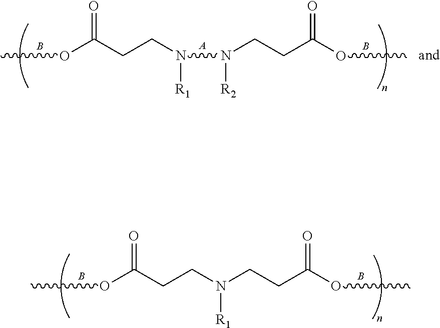
wherein:
linker A and linker B are each independently selected from the group consisting of carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups;
R₁ and R₂ are each independently selected from the group consisting of hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups; and
n is an integer greater than or equal to 5.

12. The structure of claim 10, wherein the poly(β-amino ester) is selected from the group consisting of 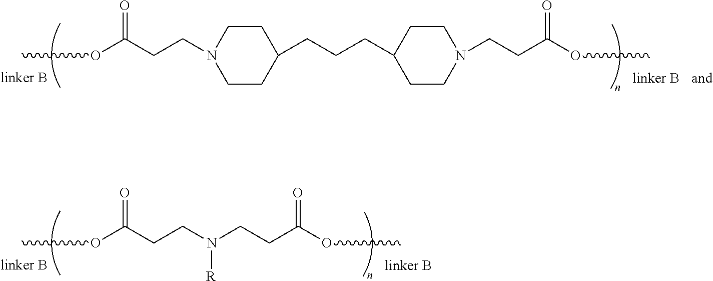
wherein:
linker B is independently selected from the group consisting of carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups;
R is selected from the group consisting of hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups; and
n is an integer greater than or equal to 5.

13. The structure of claim 10, wherein the poly(β-amino ester) is selected from the group consisting of

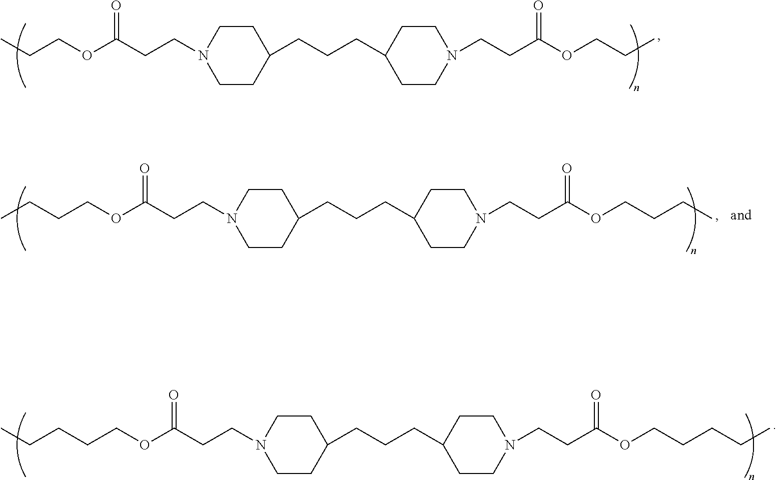

14. The structure of claim 2, wherein at least one of the first plurality of bilayers and the second plurality of bilayers comprises a polymer selected from poly(styrene sulfonate), poly(acrylic acid), linear poly(ethylene imine), poly(diallyl dimethyl ammonium chloride), poly(allylamine hydrochloride), and any combination thereof.

15. The structure of claim 2, wherein at least portion of the multilayer film comprises a biodegradable polymer.

16. The structure of claim 15, wherein the biodegradable polymer is selected from polyhydroxyacids, polypropylfumerates, polycaprolactones, polyamides, poly(amino acids), polyacetals, polyethers, biodegradable polycyanoacrylates, biodegradable polyurethanes, polysaccharides, and co-polymers, mixtures, and adducts thereof

17. The structure of claim 2, wherein at least portion of the multilayer film comprises a zwitterionic polymer.

18. The structure of claim 2, wherein at least portion of the multilayer film comprises an releasable agent selected from a group consisting of a biomolecule, a small molecule, a bioactive agent, a nanoparticle, a composite, and any combination thereof.

19. The structure of claim 2, wherein at least portion of the multilayer film comprises a therapeutic gene.

20. The structure of claim 2, wherein at least portion of the multilayer film comprises a plasmid DNA.

21. The structure of claim 1, further comprising a layer of cells.

22. The structure of claim 21, wherein the density of the cells is about or more than 5,000 cells/cm², 20,000 cells/cm², or 50,000 cells/cm².

23. The structure of claim 21, wherein the cells are selected from connective tissue cells, organ cells, muscle cells, nerve cells, stem cells, cancer cells, and any combination thereof

24. The structure of claim 21, wherein the cells are osteoblastic or pre-osteoblastic cells.

25. The structure of claim 1, further comprising a member of a cell adhesion sequence, a targeting sequence, and both disposed in the multilayer film.

26. The structure of claim 1, wherein the multilayer film is characterized by degradation selected from the group consisting of hydrolytic degradation, thermal degradation, enzymatic degradation, photolytic degradation and any combination thereof.

27. The structure of claim 1, wherein the substrate is or comprising a medical device.

28. The structure of claim 27, wherein the medical device is an implant.

29. The structure of claim 1, wherein the substrate is or comprises a microneedle substrate.

30. The structure of claim 29, wherein the microneedle substrate is a microneedle or a microneedle array.

31. A method of making comprising a step of:
forming a multilayer film on a substrate,
wherein the multilayer film comprises a first plurality of first units, each first unit comprising a protamine polypeptide.

32. (canceled)

33. A method of using comprising:
coating a substrate with a multilayer film,
wherein the multilayer film comprises a first plurality of first units, each first unit comprising a protamine polypeptide; and
wherein the multilayer film comprises a layer of cells.

34. A structure comprising:
a microneedle substrate and
a multilayer film coated on at least portion of the microneedle substrate,
wherein the multilayer film comprises an agent for release and a first plurality of
first unit; each first unit comprising a first layer and a second layer, wherein the first layer and the second layer are associated with one another.

35.-39. (canceled)

40. A method of coating a microneedle susbstrate comprising a step of:
depositing a multilayer film to at least portion of the microneedle device layer-by-layer,
wherein the multilayer film comprises an agent for release and a first plurality of first unit; each first unit comprising a first layer and a second layer, wherein the first layer and the second layer are associated with one another.

41. A method of using a coated microneedle substrate comprising a step of:
contacting the coated microneedle substrate with a biological tissue, and
releasing an agent from the coated microneedle substrate,
wherein the multilayer film comprises the agent and a first plurality of first unit;
each first unit comprising a first layer and a second layer, wherein the first layer and the second layer are associated with one another.
